(19) United States
(12) Patent Application Publication
(10) Pub. No.: US 2008/0119643 A1
(43) Pub. Date: May 22, 2008

(54) 4,4'-Diazobenzanilide Dyestuffs

(76) Inventors: Michael Lennartz, Lorrach (DE); Holger Lautenbach, Rheinfelden/Warmbach (DE)

Correspondence Address: JoAnn Villamizar;Ciba Corporation/Patent Department, 540 White Plains Road, P.O. Box 2005, Tarrytown, NY, 10591, US

(21) Appl. No.: 11/663,822

(22) PCT Filed: Oct. 10, 2005
(86) PCT No.: PCT/EP05/55118

§ 371 Date: Mar. 26, 2007

(30) Foreign Application Priority Data
Oct. 18, 2004 (EP) 04105117.8

(51) Int. Cl. C07D 403/02; C07D 401/02; C07C 245/02

(52) U.S. Cl. 534/769; 534/770; 534/816

(57) ABSTRACT

The present invention provides 4,4′-diazobenzanilide derivatives, a process for their preparation, their use as dyes, dyed paper, formulations comprising them and also precursors thereof and their processes of preparation.

The present invention refers to 4,4′-diazobenzanilide derivatives, to a process for their preparation, to their use as dyes, to dyed paper, to formulations comprising them and also to precursors thereof and their processes of preparation.

4,4′-Diazobenzanilide derivatives are common dyes.

WO 03/10433 describes 4,4′-diazobenzanilide derivatives which are derived from 4,4′-di-amino-3′-sulfobenzanilide, 4,4′-diamino-2′-methoxy-5′-sulfobenzanilide, 3,4′-diamino-3′-sulfobenzanilide, 3,4′-diamino-2′-methoxy-5′-sulfobenzanilide, 4,3′-diamino-4′-sulfobenzanilide, 3,3′-diamino-4′-sulfobenzanilide, 4,4′-diamino-2′,5′-disulfobenzanilide, 3,4′-diamino-2′,5′-disulfobenzanilide, 4,4′-diamino-3′-carboxybenzanilide or 3,4′-diamino-3′-carboxybenzanilide.

DE 2 236 250 A1 describes 4,4′-diazobenzanilide derivatives which are derived from 4,4′-diaminobenzanilide, 4,4′-diamino-2′-methoxybenzanilide, 4,4′-diamino-2′-chlorobenzanilide, 4,4′-diamino-2′-chlorobenzanilide, 4,4′-diamino-2′-methylbenzanilide or 4,4′-diamino-2′,6′-dichlorobenzanilide.

EP 0 262 095 describes 4,4′-diazobenzanilide derivatives of the formula

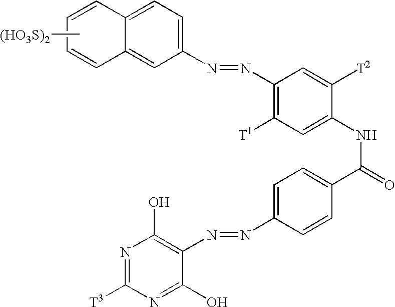

in which T¹ is hydrogen, methyl or NHCOCH₃, T² is hydrogen, methyl or methoxy, T³ is NHCN or NHCONH₂ and the sulfogroups are in 6, 8 or 5,7-position. The disadavantage of these 4,4′-diazobenzanilide derivatives is that their synthesis involves the use of toxic o-anisidine or p-cresidine derivatives.

It is an object of the present invention to provide 4,4′-diazobenzanilide derivatives, which can be used as dyes of yellow or orange shade for dyeing natural or synthetic materials, especially paper, and which can be synthesized from ecological harmless starting materials. In addition, the 4,4′-diazobenzanilide derivatives should show excellent colour strength, lightfastness and substantivity, whilst being sufficient water-soluble to be employed as an aqueous formulation.

This object is solved by the 4,4′-diazobenzanilide derivatives according to claim 1, the 4-amino-4′-azobenzanilide derivatives according to claim 2, the processes according to claims 3, 4 and 5, the paper according to claim 9 and by formulations according to claims 10 and 11.

The 4,4′-diazobenzanilide derivative of the present invention has formula

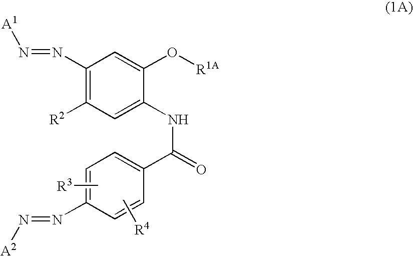

in which
A¹ represents phenyl or 1- or 2-naphthyl, whereby phenyl can be unsubstituted or mono- or disubstituted with sulfo, C₁₋₄-alkyl, C₁₋₄-alkoxy, C₂₋₄-hydroxyalkoxy, halogen, hydroxy, amino, acetamido, ureido or carboxy, and whereby 1- or 2-naphthyl can be unsubstituted or substituted with one or more sulfo groups, and

### A² represents a residue selected from the group consisting of

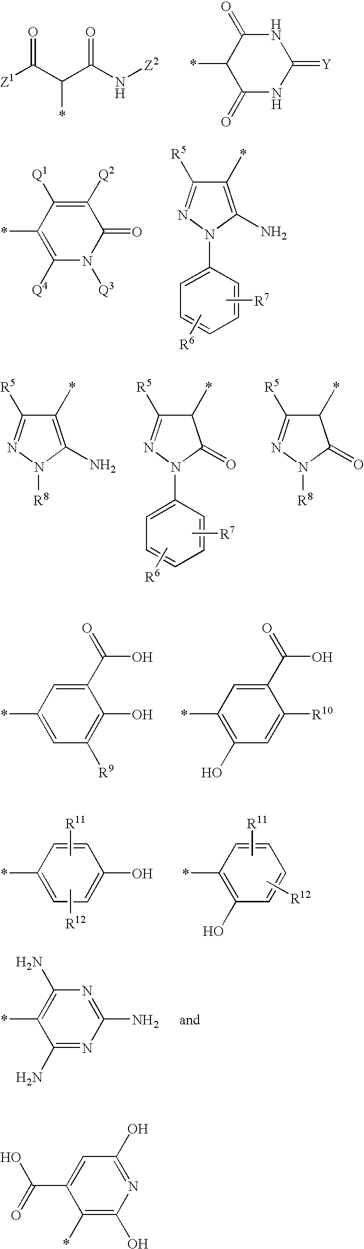

in which

### Z¹ represents C₁₋₄-alkyl or phenyl, whereby phenyl may be unsubstituted or mono-substituted with C₁₋₄-alkyl, C₁₋₄-alkoxy or halogen, and

Z² represents phenyl or 1- or 2-naphthyl, whereby phenyl may be unsubstituted or mono-, di- or trisubstituted with sulfo, C₁₋₄-alkyl, C₁₋₄-alkoxy, C₂₋₄-hydroxyalkoxy, halogen, hydroxy, amino, acetamido, ureido or carboxy and whereby 1- or 2-naphthyl may be unsubstituted or mono- or disubstituted with sulfo or carboxy,

### Y represents O, N—CN or N—CONH₂,

### Q¹ represents hydrogen, hydroxy, C₁₋₂-alkyl, hydroxyethyl, C₁₋₂-alkoxy, carboxy, carbamoyl, C₁₋₂-alkoxycarbonyl, and

### Q² represents hydrogen, cyano, halogen, sulfo, C₁₋₂-alkyl or carbamoyl, whereby C₁₋₂-alkyl may be unsubstituted or substituted with hydroxy, phenyl or sulfo, and

### Q³ represents hydrogen, phenyl, C₁₋₂-alkylphenyl, C₁₋₄-alkyl, whereby C₁₋₄-alkyl may be unsubstituted or substituted with hydroxy, cyano, C₁₋₂-alkoxy or sulfo, and

### Q⁴ represents hydrogen or hydroxy,

### R⁵ represents hydrogen, C₁₋₄-alkyl, C₂₋₄-alkenyl, carboxy, NHCOC₁₋₄-alkyl, and

### R⁶ and R⁷ each independently from each other represent hydrogen, halogen, sulfo, C₁₋₄-alkyl or carboxy, and

### R⁸ represents hydrogen or C₁₋₄-alkyl,

### R⁹ represents hydrogen, C₁₋₄-alkyl, and

### R¹⁰ represents hydrogen or hydroxy,

### R¹¹ and R¹² each independently from each other represent hydrogen, C₁₋₄-alkyl, C₁₋₄-alkoxy, hydroxy, halogen, amino, acetamido, sulfo, carboxy, C₁₋₄-alkoxycarbonyl or C₁₋₄-alkylaminocarbonyl, and

### R² represents hydrogen, C₁₋₄-alkyl, C₁₋₄-alkoxy, halogen, hydroxy, carboxy, acetamido, ureido or sulfo, whereby C₁₋₄-alkyl and C₁₋₄-alkoxy may be unsubstituted or substituted by halogen, hydroxy, carboxy, acetamido, ureido or sulfo, and

R³ and R⁴ each independently from each other represent hydrogen, C₁₋₄-alkyl, C₁₋₄-alkoxy, halogen, hydroxy, carboxy, amino, C₁₋₄-alkylamino, acetamido or ureido, whereby C₁₋₄-alkyl and C₁₋₄-alkoxy may be unsubstituted or substituted by halogen, hydroxy, carboxy, amino, C₁₋₄-alkylamino, acetamido or ureido, and

### R^{1A} represents a residue selected from the group consisting of

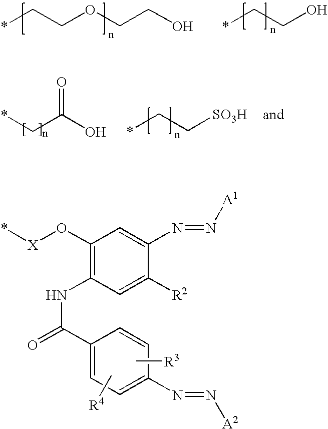

in which
n≧1,

### A¹, A², R², R³ and R⁴ have the meaning as indicated above, and

### X represents C₂₋₁₄-alkylene, whereby a —CH₂CH₂CH₂— unit of C₂₋₁₄-alkylene may be replaced by a —CH₂-E-CH₂— unit, in which E represents O, NH or S.

C₁₋₄-Alkyl can be methyl, ethyl, propyl, isopropyl, butyl, sec-butyl, tert-butyl or isobutyl. C₁₋₄-alkoxy can be methoxy, ethoxy, propoxy, isopropoxy, butoxy, sec-butoxy, tert-butoxy or isobutoxy. C₂₋₄-hydroxyalkoxy can be 2-hydroxyethoxy, 3-hydroxypropoxy, 2-hydroxypropoxy, 1-hydroxyisopropoxy or 4-hydroxybutoxy. Halogen can be fluorine, bromine, chlorine or iodine. C₁₋₂-alkyl is methyl or ethyl. C₁₋₂-alkoxy is methoxy or ethoxy. C₁₋₂-alkoxycarbonyl is methoxycarbonyl or ethoxycarbonyl. C₁₋₂-alkylphenyl can be o-, m- or p-tolyl or 2-, 3-, or 4-ethylphenyl. C₂₋₄-alkenyl can be vinyl, 1-propenyl, allyl, 1-butenyl or 2-butenyl. NHCOC₁₋₄-alkyl can be acetamido, propionylamino or butyrylamino. C₁₋₄-alkylaminocarbonyl can be methylaminocarbonyl, ethylaminocarbonyl, propylaminocarbonyl, butylaminocarbonyl, tert-butylaminocarbonyl or isobutylaminocarbonyl. C₁₋₄-Alkylamino can be methylamino, ethylamino, propylamino, isopropylamino, butylamino, sec-butylamino, tert-butylamino or isobutylamino. C₂₋₁₄-alkylene can be ethylene, trimethylene, propylene, tetramethylene, ethylethylene, pentamethylene, hexamethylene, heptamethylene or octamethylene. Examples of a C₂₋₁₄-alkylene, whereby a —CH₂CH₂CH₂— unit of C₂₋₁₄-alkylene may be replaced by a —CH₂-E-CH₂— unit, in which E represents O, are —CH₂CH₂—O—CH₂CH₂—O—CH₂CH₂—, —CH₂CH₂—O—CH₂CH₂—, —CH₂CH₂—O—CH₂CH₂—O—CH₂CH₂—O—CH₂CH₂— and —CH₂CH₂—O—CH₂CH₂—O—CH₂CH₂—O—CH₂CH₂—O—CH₂CH₂—.

In preferred 4,4′-diazobenzanilide derivatives 1A

A¹ represents phenyl or 1- or 2-naphthyl, whereby phenyl and 1- or 2-naphthyl are substituted with at least one sulfo group, and whereby phenyl may additionally be mono-substituted with C₁₋₄-alkyl, C₁₋₄-alkoxy, C₂₋₄-hydroxyalkoxy, halogen, hydroxy, acetamido, ureido or carboxy, and

### A² represents a residue selected from the group consisting of

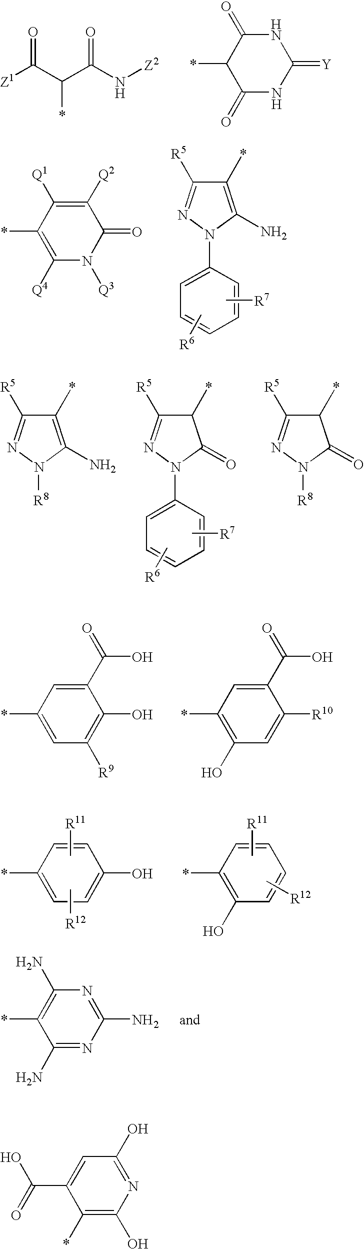

in which

### Z¹ represents C₁₋₄-alkyl or phenyl, whereby phenyl may be unsubstituted or mono-substituted with C₁₋₄-alkyl, C₁₋₄-alkoxy or halogen, and

Z² represents phenyl or 1- or 2-naphthyl, whereby phenyl may be unsubstituted or mono-, di- or trisubstituted with sulfo, C₁₋₄-alkyl, C₁₋₄-alkoxy, C₂₋₄-hydroxyalkoxy, halogen, hydroxy, amino, acetamido, ureido or carboxy and whereby 1- or 2-naphthyl may be unsubstituted or mono- or disubstituted with sulfo or carboxy,

### Y represents O, N—CN or N—CONH₂,

### Q¹ represents hydrogen, hydroxy, C₁₋₂-alkyl, hydroxyethyl, C₁₋₂-alkoxy, carboxy, carbamoyl, C₁₋₂-alkoxycarbonyl, and

Q² represents hydrogen, cyano, halogen, sulfo, C₁₋₂-alkyl or carbamoyl, whereby C₁₋₂-alkyl may be unsubstituted or substituted with hydroxy, phenyl or sulfo, and

### Q³ represents hydrogen, phenyl, C₁₋₂-alkylphenyl, C₁₋₄-alkyl, whereby C₁₋₄-alkyl may be unsubstituted or substituted with hydroxy, cyano, C₁₋₂-alkoxy or sulfo, and

### Q⁴ represents hydrogen or hydroxy,

### R⁵ represents hydrogen, C₁₋₄-alkyl, C₂₋₄-alkenyl, carboxy, NHCOC₁₋₄-alkyl, and

### R⁶ and R⁷ each independently from each other represent hydrogen, halogen, sulfo, C₁₋₄-alkyl or carboxy, and

### R⁸ represents hydrogen or C₁₋₄-alkyl,

### R⁹ represents hydrogen, C₁₋₄-alkyl, and

### R¹⁰ represents hydrogen or hydroxy,

### R¹¹ and R¹² each independently from each other represent hydrogen, C₁₋₄-alkyl, C₁₋₄-alkoxy, hydroxy, halogen, amino, acetamido, sulfo, carboxy, C₁₋₄-alkoxycarbonyl or C₁₋₄-alkylaminocarbonyl, and

### R² represents hydrogen, C₁₋₄-alkyl, C₁₋₄-alkoxy, halogen, carboxy or sulfo,

### R³ and R⁴ each independently from each other represent hydrogen or C₁₋₄-alkyl,

### R^{1A} represents a residue selected from the group consisting of

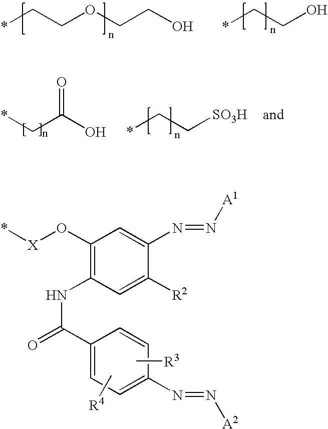

in which
n≧1,

### A¹, A², R², R³ and R⁴ have the meaning as indicated for the preferred 4,4′-diazobenzanilide derivatives 1A, and X represents C₂₋₁₄-alkylene, whereby a —CH₂CH₂CH₂— unit of C₂₋₁₄-alkylene may be replaced by a —CH₂-E-CH₂— unit, in which E represents O, NH or S.

In more preferred 4,4′-diazobenzanilide derivatives 1A

### A¹ represents phenyl or 2-naphthyl, whereby phenyl and 2-naphthyl are substituted with at least one sulfo group, and whereby phenyl may additionally be mono-substituted with C₁₋₄-alkyl or C₁₋₄-alkoxy, and

### A² represents a residue selected from the group consisting of

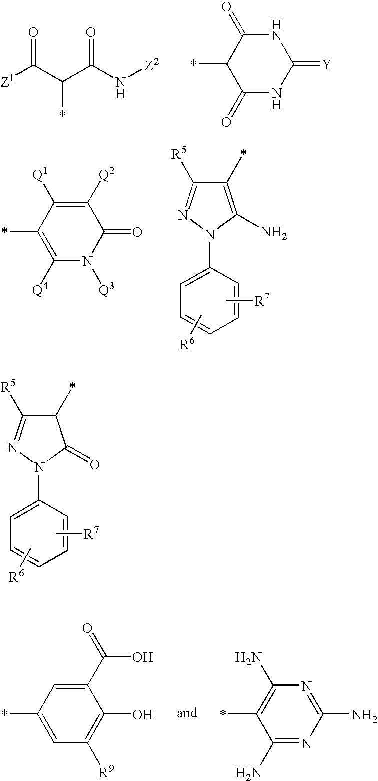

in which

### Z¹ represents C₁₋₄-alkyl,

### Z² represents phenyl, whereby phenyl may be unsubstituted or mono-, di- or trisubstituted with sulfo, C₁₋₄-alkyl or C₁₋₄-alkoxy,

### Y represents O or N—CN,

### Q¹ represents hydrogen or C₁₋₂-alkyl,

### Q² represents cyano, C₁₋₂-alkyl or carbamoyl, whereby C₁₋₂-alkyl may be unsubstituted or substituted with sulfo,

### Q³ represents C₁₋₄-alkyl,

### Q⁴ represents hydroxy,

### R⁵ represents hydrogen or C₁₋₄-alkyl, R⁶ and R⁷ each independently from each other represent hydrogen, sulfo or C₁₋₄-alkyl,

### R⁹ represents hydrogen or C₁₋₄-alkyl,

### R² represents hydrogen or C₁₋₄-alkyl,

### R³ and R⁴ each independently from each other represent hydrogen or C₁₋₄-alkyl,

### R^{1A} represents a residue selected from the group consisting of

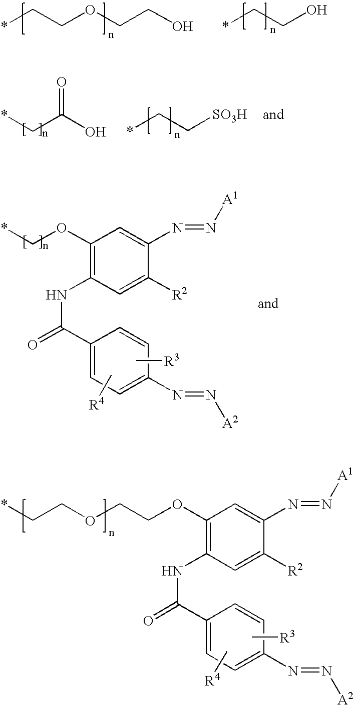

in which
n≧1,
and A¹, A², R², R³ and R⁴ have the meaning as indicated above for the more preferred 4,4′-diazobenzanilide derivatives 1A.

In even more preferred 4,4′-diazobenzanilide derivatives 1A

### A¹ represents phenyl or 2-naphtyl, whereby phenyl is substituted with at least one sulfo group and 2-naphthyl is substituted with at least two sulfo groups, and

### A² represents a residue selected from the group consisting of

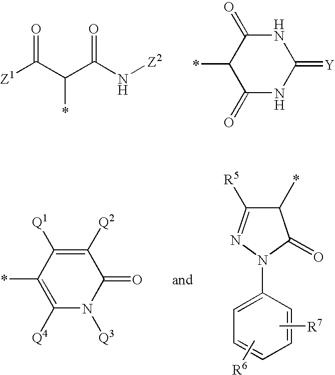

in which

### Z¹ represents C₁₋₄-alkyl,

### Z² represents phenyl, whereby phenyl may be unsubstituted or mono-, di- or trisubstituted with sulfo, C₁₋₄-alkyl or C₁₋₄-alkoxy,

### Y represents O or N—CN,

### Q¹ represents hydrogen C₁₋₂-alkyl,

### Q² represents cyano,

### Q³ represents C₁₋₄-alkyl,

### Q⁴ represents hydroxy,

### R⁵ represents C₁₋₄-alkyl,

### R⁶ and R⁷ represent hydrogen,

### R² represents hydrogen or C₁₋₄-alkyl, and

### R³ and R⁴ represent hydrogen, and

### R^{1A} represents a residue selected from the group consisting of

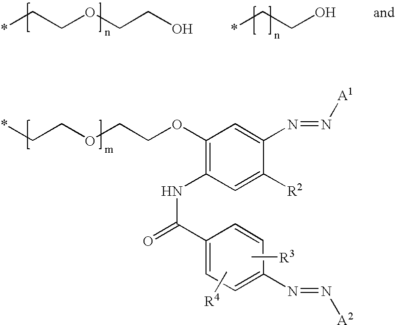

in which
n≧1,
m≧0,
and A¹, A², R², R³ and R⁴ have the meaning as indicated above for the even more preferred 4,4′-diazobenzanilide derivatives 1A.

In most preferred 4,4′-diazobenzanilide derivatives 1A

### A¹ represents 4-sulfophenyl, 6,8-disulfo 2-naphthyl or 4,8-disulfo 2-naphthyl, and

### A² represents a residue selected from the group consisting of

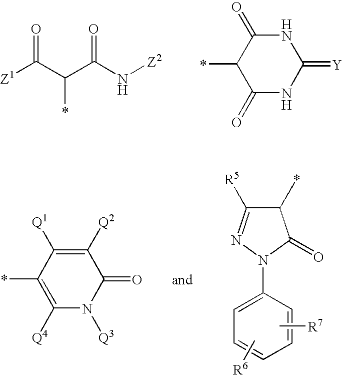

in which

### Z¹ represents methyl,

### Z² represents 5-methyl-2-methoxy-4-sulfophenyl

### Y represents O or N—CN,

### Q¹ represents methyl,

### Q² represents cyano,

### Q³ represents ethyl,

### Q⁴ represents hydroxy,

### R⁵ represents methyl

### R⁶ and R⁷ represent hydrogen,

### R² represents hydrogen or methyl, and

### R³ and R⁴ represent hydrogen, and

### R^{1A} represents a residue selected from the group consisting of 2-hydroxyethyl and

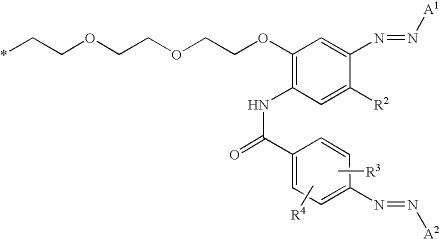

in which

### A¹, A², R², R³ and R⁴ have the meaning as indicated above for the most preferred 4,4′-diazobenzanilide derivatives 1A.

Also part of the invention is the 4-amino-4′-azobenzanilide derivative of the formula

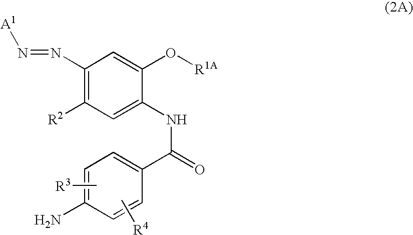

in which A¹, R², R³ and R⁴ have the meaning as indicated above, and

### R^{1A} represents a residue selected from the group consisting of

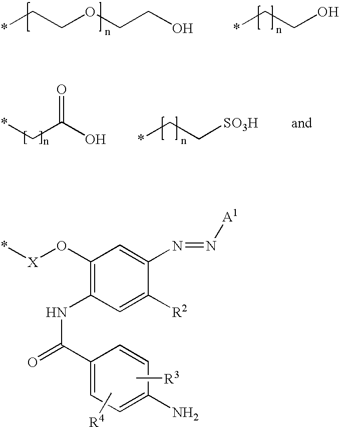

in which
n≧1,

### A¹, R², R³ and R⁴ have the meaning as indicated above, and

### X represents C₂₋₁₄-alkylene, whereby a —CH₂CH₂CH₂— unit of C₂₋₁₄-alkylene may be replaced by a —CH₂-E-CH₂— unit, in which E represents O, NH or S.

The process of the present invention for the preparation of 4-amino-4′-azobenzanilide derivative of the formula

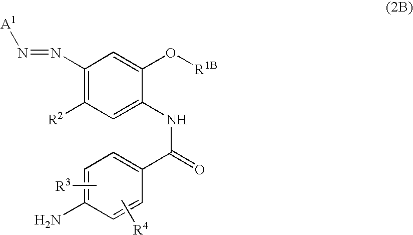

in which A¹, R², R³ and R⁴ have the meaning as indicated above, and

### R^{1B} represents a residue selected from the group consisting of

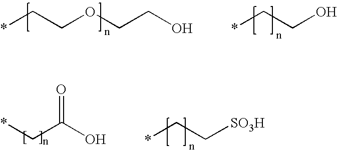

in which
n≧1,
comprises the steps of
i) reacting a 2-nitrophenol derivative of the formula

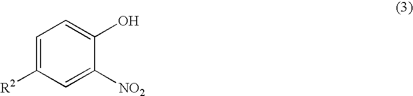

with a compound of the formula

R^{1B}-LG (4B)
in which LG represents a leaving group, to yield a nitrobenzol derivative of the formula

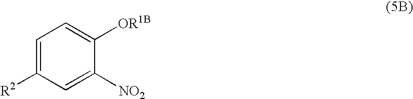

ii) reducing the nitrobenzol derivative of formula 5B obtained in step i) to yield an aniline derivative of the formula

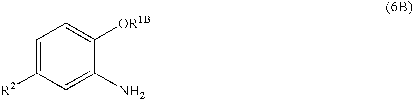

iii) diazotizing an amine of the formula

A¹-NH₂ (7)
to yield a diazonium ion of the formula

A¹—N⁺≡N (8)
iv) coupling the diazonium ion of the formula 8 obtained in step iii) with the aniline derivative of formula 6B obtained in step ii) to yield a coupling product of the formula

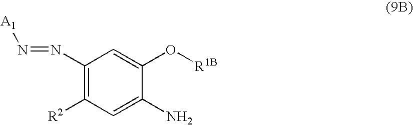

v) reacting the coupling product of formula 9B obtained in step iv) with a nitrobenzoylchloride derivative of the formula

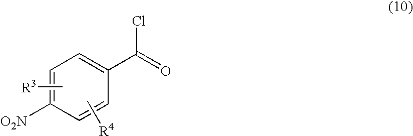

to yield a nitro compound of the formula

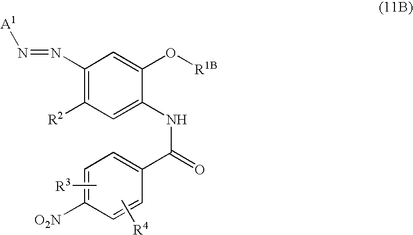

vi) reducing the nitro compound of the formula 11B obtained in step v) to yield the 4-amino-4′-azobenzanilide derivative of formula 2B.

Leaving group can be those functionalities typically used in the synthesis of alkylarylethers via Williamson synthesis, e.g. halogen, sulfate or arylsulfonate.

The process of the present invention for the preparation of 4-amino-4′-azobenzanilide derivative of the formula

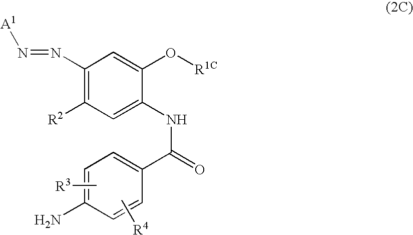

in which A¹, R², R³ and R⁴ have the meaning as indicated above, and R^{1C} represents

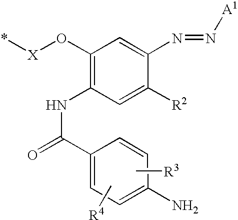

in which

### A¹, R², R³ and R⁴ have the meaning as indicated above, and

### X represents C₂₋₁₄-alkylene, whereby a —CH₂CH₂CH₂— unit of C₂₋₁₄-alkylene may be replaced by a —CH₂-E-CH₂— unit, in which E represents O, NH or S,

comprises the steps of
i) reacting a 2-nitrophenol derivative of the formula

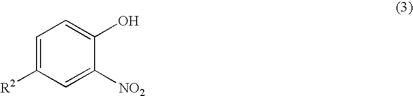

with a compound of the formula

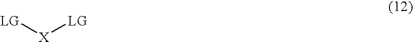

in which LG represents a leaving group, to yield a nitrobenzol derivative of the formula

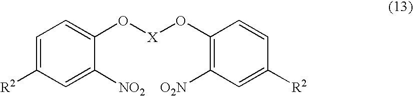

ii) reducing the nitrobenzol derivative of formula 13 obtained in step i) to yield an aniline derivative of the formula

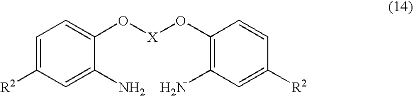

iii) diazotizing an amine of the formula

A¹-NH₂ (7)
to yield a diazonium ion of the formula

A¹—N⁺≡N (8)
iv) coupling the diazonium ion of the formula 8 obtained in step iii) with the aniline derivative of formula 14 obtained in step ii) to yield a coupling product of the formula

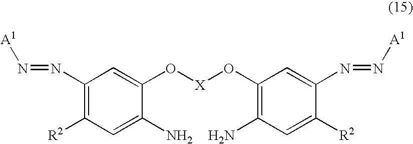

v) reacting the coupling product of formula 15 obtained in step iv) with a nitrobenzoylchloride derivative of the formula

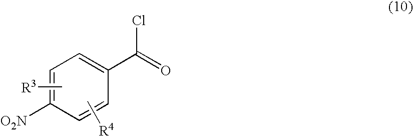

to yield a nitro compound of the formula

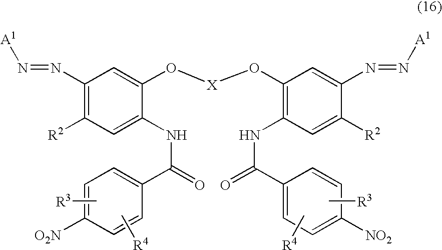

vi) reducing the nitro compound of the formula 16 obtained in step v) to yield the 4-amino-4′-azobenzanilide derivative of formula 2C.

The process of the present invention for the preparation of 4,4′-diazobenzanilide derivative of the formula

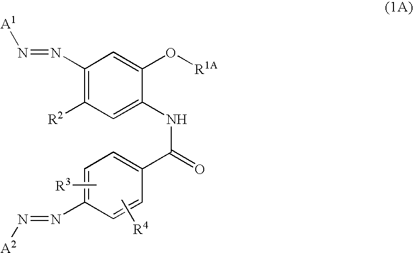

in which A¹, A², R², R³ and R⁴ have the meaning as indicated above and R^{1A} represents a residue selected from the group consisting of

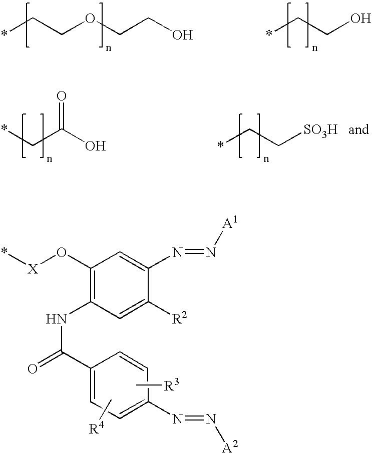

in which
n≧1,

### A¹, A², R², R³ and R⁴ have the meaning as indicated above, and

### X represents C₂₋₁₄-alkylene, whereby a —CH₂CH₂CH₂— unit of C₂₋₁₄-alkylene may be replaced by a —CH₂-E-CH₂— unit, in which E represents O, NH or S.

comprises the steps of
i) diazotizing a 4-amino-4′-azobenzanilide derivative of the formula

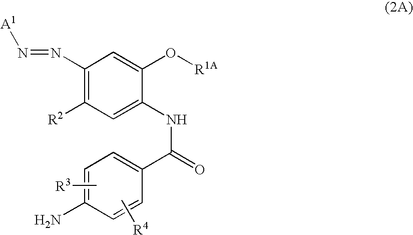

to yield a diazonium ion of the formula

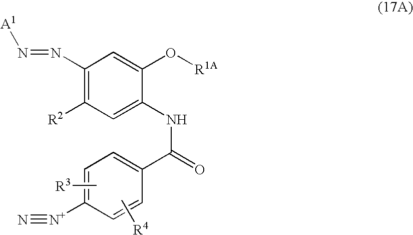

in which A¹, R², R³ and R⁴ have the meaning as indicated above and R^{1A} represents a residue selected from the group consisting of

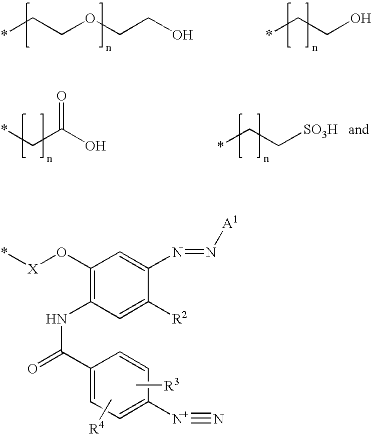

in which
n≧1,
A¹, A², R², R³ and R⁴ have the meaning as indicated above, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S,
ii) coupling the diazonium ion 17A obtained in step i) with a compound of the formula

A²-H (18)
in which A² has the meaning as indicated above to yield the 4,4′-diazobenzanilide derivative 1A.

Preferably, the 4-amino-4′-azobenzanilide derivative is prepared according to one of the above processes of the present invention.

A₁-NH₂ and A²-H are known compounds or may be prepared by known methods.

The 4,4′-diazobenzanilide derivatives 1A can be used for dyeing natural or synthetic materials such as paper, cellulose, polyamide, leather or glass fibres. Preferably, the 4,4′-diazobenzanilide derivatives 1A are used for dyeing paper.

Paper dyed with the 4,4′-diazobenzanilide derivatives 1A is also part of the invention.

The 4,4′-diazobenzanilide derivatives 1A can be applied to the materials, preferably to paper, in the form of aqueous or solid formulations.

The aqueous and solid formulations comprising 4,4′-diazobenzanilide derivatives 1A are also part of the invention.

The solid formulations comprising 4,4′-diazobenzanilide derivatives 1A can be powders or granulate materials, and may include auxiliaries. Examples of auxiliaries are solubilizers such as urea, extenders such as dextrin, Glauber salt or sodium chloride, sequestrants such as tetrasodium phosphate, and also dispersants and dustproofing agents.

The aqueous formulations comprising 4,4′-diazobenzanilide derivatives 1A may also include auxiliaries. Examples of auxiliaries used for aqueous formulations are solubilizers such as ε-caprolactam or urea, and organic solvents such as glycols, polyethylene glycols, dimethyl sulphoxide, N-methylpyrrolidone, acetamide, alkanolamines or polyglycolamines.

Preferably, the aqueous formulations are aqueous solutions which comprise from 5 to 30% by weight 4,4′-diazobenzanilide derivatives 1A based on the weight of the solution. Preferably, these concentrated aqueous solutions also contain a low level of inorganic salts, which may be achieved by known methods, for example by reverse osmosis.

Aqueous formulations of the 4,4′-diazobenzanilide derivatives 1A can also be used for the preparation of inks.

The 4,4′-diazobenzanilide derivatives 1A are dyes of yellow or orange shade, which can be synthesized from ecological harmless starting materials, and which show a good brilliance, a high substantivity, a high degree of exhaustion and a good to very good lightfastness.

### EXAMPLES

### Example 1

### Preparation of the 4-amino-4′-azobenzanilide derivative of the formula

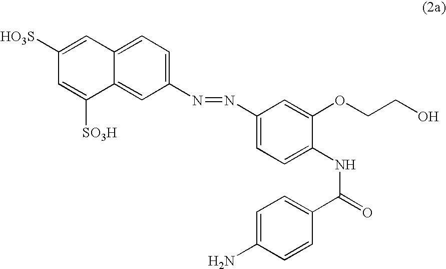

### (A¹ is 6,8-disulfo-2-naphthyl, R², R³ and R⁴ are hydrogen and R^{1A}, respectively, R^{1B} is 2-hydroxyethyl)

Ethylene chlorohydrin (143.2 g) is added to a solution of 2-nitrophenol (139.11 g) in water (225 g) at 75 to 80° C. and at pH 8.8 to 9.3 within 30 minutes. The reaction mixture is stirred overnight, aqueous ammonia (25 w %, 34 g) is added and the reaction mixture is stirred for further 30 minutes. The organic layer containing the nitrobenzol derivative 5a (R^{1B} is 2-hydroxyethyl, R² is hydrogen) is separated, diluted with a mixture of ethanol/water (1/3.7, 1400 mL) and heated to 85 to 90° C. Sodium sulfide (141.8 g) is added and the reaction mixture is stirred until the reaction was complete. The reaction mixture is cooled to room temperature and concentrated. The obtained suspension is filtered and the filter cake is dried in vacuo to yield 135.5 g of the aniline derivative 6a (R^{1B} is 2-hydroxyethyl, R² is hydrogen).

Aqueous HCl (32 w %, 35 g) is added to a suspension of 2-naphthylamine-6,8-disulfonic acid (36.9 g) in water (300 mL) at 5 to 10° C., followed by addition of sodium nitrite (4 N, 32 mL) within 40 minutes. The reaction mixture is stirred for 1 hour, and then unreacted nitrite is destroyed by addition of sulfamic acid. A suspension obtaining the diazonium ion 8a (A¹ is 6,8-disulfo-2-naphthyl) is obtained.

This suspension is added to a suspension of the aniline derivative 6a (18.9 g) in water (300-mL) at pH 4.5 to 5.0 within 30 minutes. The reaction mixture is stirred at pH 4.5 to 5.0 until the reaction is complete. The reaction mixture is concentrated and treated with sodium chloride. The resulting suspension is filtered and the filter cake is dried in vacuo to yield 53.7 g of the coupling product 9a (A¹ is 6,8-disulfo-2-naphthyl, R^{1B} is 2-hydroxyethyl, R² is hydrogen).

A solution of 4-nitrobenzoylchloride (5.7 g) in acetone (50 mL) is added to a suspension of the coupling product 9a (13 g) in water (150 g) at below 32° C. and at pH 6.5 to 7.0. The reaction mixture is stirred overnight, filtered and the filter cake is dried in vacuo to yield 13.7 g of the nitro compound 11a (A¹ is 6,8-disulfo-2-naphthyl, R^{1B} is 2-hydroxyethyl, R², R³ and R⁴ are hydrogen).

Aqueous sodium sulfide (60 w %, 4.8 g) is added to a suspension of the nitro compound 11a (13 g) in water (80 g) at 50° C. The reaction mixture is stirred at 50 to 55° C. for 1 hour, treated with sodium chloride and concentrated. The resulting suspension is filtered and the filter cake is dried in vacuo to yield 8.9 g of the 4-amino-4′-azobenzanilide derivative of the formula 2a.

### Examples 2 to 60

### Preparation of the 4-amino-4′-azobenzanilide derivative of the formula

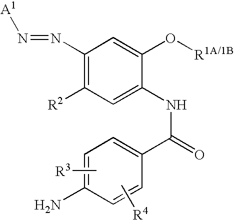

### (2A, respectively, 2B)

in which R³ and R⁴ are hydrogen, and

TABLE 1

| Example No/ | | | |
|---|---|---|---|
| Compound No | A¹ | R^{1A}, respectively, R^{1B} | R² |
| 2/2b | 6,8-disulfo-2-naphthyl | 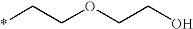 | hydrogen |
| 3/2c | 6,8-disulfo-2-naphthyl | 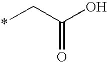 | hydrogen |
| 4/2d | 6,8-disulfo-2-naphthyl | 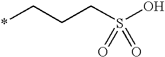 | hydrogen |
| 5/2e | 6,8-disulfo-2-naphthyl | 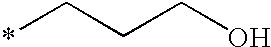 | hydrogen |
| 6/2f | 6,8-disulfo-2-naphthyl | 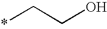 | methyl |
| 7/2g | 6,8-disulfo-2-naphthyl | 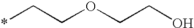 | methyl |
| 8/2h | 6,8-disulfo-2-naphthyl | 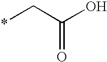 | methyl |
| 9/2i | 6,8-disulfo-2-naphthyl | 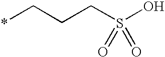 | methyl |
| 10/2j | 6,8-disulfo-2-naphthyl | 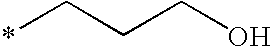 | methyl |
| 11/2k | 4,8-disulfo-2-naphthyl | 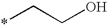 | hydrogen |
| 12/2l | 4,8-disulfo-2-naphthyl | 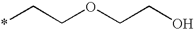 | hydrogen |
| 13/2m | 4,8-disulfo-2-naphthyl | 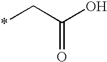 | hydrogen |
| 14/2n | 4,8-disulfo-2-naphthyl | 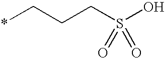 | hydrogen |
| 15/2o | 4,8-disulfo-2-naphthyl | 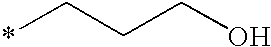 | hydrogen |
| 16/2p | 4,8-disulfo-2-naphthyl | 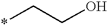 | methyl |
| 17/2q | 4,8-disulfo-2-naphthyl | 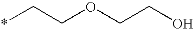 | methyl |
| 18/2r | 4,8-disulfo-2-naphthyl | 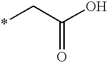 | methyl |
| 19/2s | 4,8-disulfo-2-naphthyl | 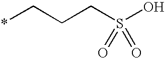 | methyl |
| 20/2t | 4,8-disulfo-2-naphthyl | 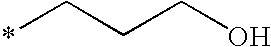 | methyl |
| 21/2u | 3,6-disulfo-2-naphthyl | 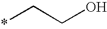 | hydrogen |
| 22/2v | 3,6-disulfo-2-naphthyl | 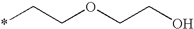 | hydrogen |
| 23/2w | 3,6-disulfo-2-naphthyl | 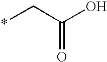 | hydrogen |
| 24/2x | 3,6-disulfo-2-naphthyl | 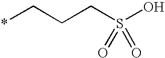 | hydrogen |
| 25/2y | 3,6-disulfo-2-naphthyl | 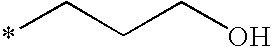 | hydrogen |
| 26/2z | 3,6-disulfo-2-naphthyl | 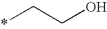 | methyl |
| 27/2aa | 3,6-disulfo-2-naphthyl | 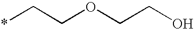 | methyl |
| 28/2ab | 3,6-disulfo-2-naphthyl | 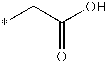 | methyl |
| 29/2ac | 3,6-disulfo-2-naphthyl | 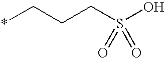 | methyl |
| 30/2ad | 3,6-disulfo-2-naphthyl | 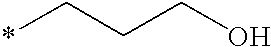 | methyl |
| 31/2ae | 5,7-disulfo-2-naphthyl | 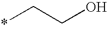 | hydrogen |
| 32/2af | 5,7-disulfo-2-naphthyl | 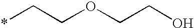 | hydrogen |
| 33/2ag | 5,7-disulfo-2-naphthyl | 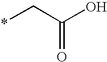 | hydrogen |
| 34/2ah | 5,7-disulfo-2-naphthyl | 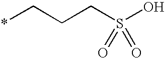 | hydrogen |
| 35/2ai | 5,7-disulfo-2-naphthyl | 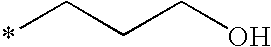 | hydrogen |
| 36/2aj | 5,7-disulfo-2-naphthyl | 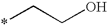 | methyl |
| 37/2ak | 5,7-disulfo-2-naphthyl | 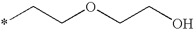 | methyl |
| 38/2al | 5,7-disulfo-2-naphthyl | 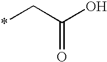 | methyl |
| 39/2am | 5,7-disulfo-2-naphthyl | 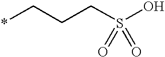 | methyl |
| 40/2an | 5,7-disulfo-2-naphthyl | 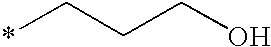 | methyl |
| 41/2ao | 1,5-disulfo-2-naphthyl | 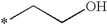 | hydrogen |
| 42/2ap | 1,5-disulfo-2-naphthyl | 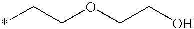 | hydrogen |
| 43/2aq | 1,5-disulfo-2-naphthyl | 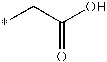 | hydrogen |
| 44/2ar | 1,5-disulfo-2-naphthyl | 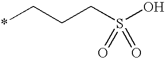 | hydrogen |
| 45/2as | 1,5-disulfo-2-naphthyl | 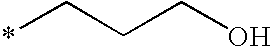 | hydrogen |
| 46/2at | 1,5-disulfo-2-naphthyl | 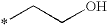 | methyl |
| 47/2au | 1,5-disulfo-2-naphthyl | 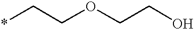 | methyl |
| 48/2av | 1,5-disulfo-2-naphthyl | 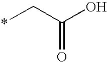 | methyl |
| 49/2aw | 1,5-disulfo-2-naphthyl | 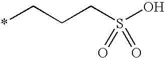 | methyl |
| 50/2ax | 1,5-disulfo-2-naphthyl | 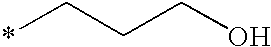 | methyl |
| 51/2ay | 1,6-disulfo-2-naphthyl | 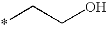 | hydrogen |
| 52/2az | 1,6-disulfo-2-naphthyl | 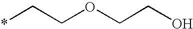 | hydrogen |
| 53/2ba | 1,6-disulfo-2-naphthyl | 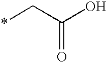 | hydrogen |
| 54/2bb | 1,6-disulfo-2-naphthyl | 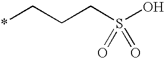 | hydrogen |
| 55/2bc | 1,6-disulfo-2-naphthyl | 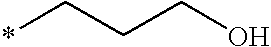 | hydrogen |
| 56/2bd | 1,6-disulfo-2-naphthyl | 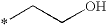 | methyl |
| 57/2be | 1,6-disulfo-2-naphthyl | 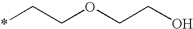 | methyl |
| 58/2bf | 1,6-disulfo-2-naphthyl | 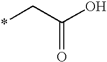 | methyl |
| 59/2bg | 1,6-disulfo-2-naphthyl | 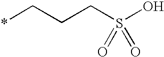 | methyl |
| 60/2bh | 1,6-disulfo-2-naphthyl | 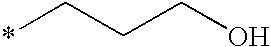 | methyl |

These 4-amino-4′-azobenzanilide derivative are prepared in analogy to example 1.

### Example 61

### Preparation of the 4-amino-4′-azobenzanilide derivative of the formula

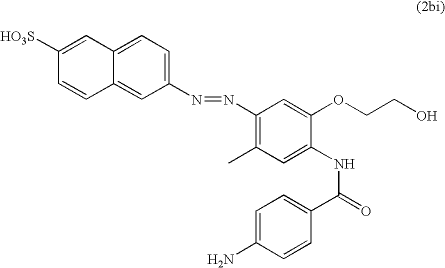

### (A¹ is 6-sulfo-2-naphthyl, R² is methyl, R³ and R⁴ are hydrogen and R^{1A}, respectively, R^{1B}, is 2-hydroxyethyl)

Ethylene chlorohydrin (120.8 g) is added to a solution of 4-methyl-2-nitrophenol (153.1 g) in water (225 g) at 75 to 80° C. and at pH 8.8 to 9.3 within 5 minutes. The reaction mixture is stirred overnight, aqueous ammonia (25 w %, 34 g) is added and the reaction mixture is stirred for further 30 minutes. The organic layer containing the nitrobenzol derivative 5b (R^{1B} is 2-hydroxyethyl, R² is methyl) is separated, diluted with isopropanol (22 mL) and heated to 85 to 90° C. Sodium sulfide (132.6 g) in 220 g of water is added slowly and the reaction mixture is stirred until the reaction was complete. The reaction mixture is cooled to room temperature. The obtained suspension is filtered and the filter cake is dried in vacuo to yield 137 g of the aniline derivative 6b (R^{1B} is 2-hydroxyethyl, R² is methyl).

Aqueous HCl (32 w %, 28.5 g) is added to a suspension of 2-naphthylamine-6-sulfonic acid (22.3 g) in water (300 mL) at 5 to 10° C., followed by addition of sodium nitrite (4 N, 25.5 mL) within 40 minutes. The reaction mixture is stirred for 1 hour, and then unreacted nitrite is destroyed by addition of sulfamic acid. A suspension obtaining the diazonium ion 8b (A¹ is 6-sulfo-2-naphthyl) is obtained.

This suspension is added to a suspension of the aniline derivative 6b (R^{1B} is 2-hydroxyethyl, R² is methyl) (17 g) in water (100 mL) at pH 3.0 to 3.8 within 30 minutes. The reaction mixture is stirred at pH 3.0 to 3.8 until the reaction is complete, stirred overnight, filtered and the filter cake is dried in vacuo to yield 40 g of the coupling product 9b (A¹ is 6-sulfo-2-naphthyl, R^{1B} is 2-hydroxyethyl, R² is methyl).

A solution of 4-nitrobenzoylchloride (12.15 g) in acetone (75 mL) is added to a suspension of the coupling product 9b (25 g) in water (150 g) at below 32° C. and at pH 6.5 to 7.0. The reaction mixture is stirred overnight, filtered and the filter cake is dried in vacuo to yield 31.3 g of the nitro compound 11b (A¹ is 6-sulfo-2-naphthyl, R^{1B} is 2-hydroxyethyl, R² is methyl, R³ and R⁴ are hydrogen).

Aqueous sodium sulfide (60 w %, 4.8 g) is added to a suspension of the nitro compound 11b (10 g) in water (80 g) at 50° C. The reaction mixture is stirred at 50 to 55° C. for 1 hour, filtered and the filter cake is dried in vacuo to yield 8.6 g of the 4-amino-4′-azobenzanilide derivative of the formula 2bi.

### Example 62

### Preparation of the 4-amino-4′-azobenzanilide derivative of the formula

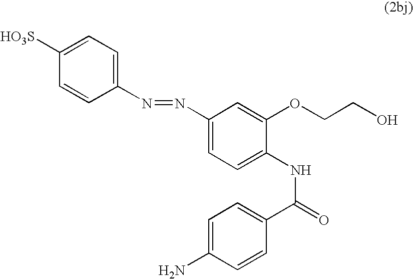

### (A¹ is 4-sulfophenyl, R², R³ and R⁴ are hydrogen and R^{1A}, respectively, R^{1B} is 2-hydroxyethyl).

Ethylene chlorohydrin (143.2 g) is added to a solution of 2-nitrophenol (139.11 g) in water (225 g) at 75 to 80° C. and at pH 8.8 to 9.3 within 30 minutes. The reaction mixture is stirred overnight, aqueous ammonia (25 w %, 34 g) is added and the reaction mixture is stirred for further 30 minutes. The organic layer containing the nitrobenzol derivative 5a (R^{1B} is 2-hydroxyethyl, R² is hydrogen) is separated, diluted with a mixture of ethanol/water (1/3.7, 1400 mL) and heated to 85 to 90° C. Sodium sulfide (141.8 g) is added and the reaction mixture is stirred until the reaction is complete. The reaction mixture is cooled to room temperature and concentrated. The obtained suspension is filtered and the filter cake is dried in vacuo to yield 135.5 g of the aniline derivative 6a (R^{1B} is 2-hydroxyethyl, R² is hydrogen).

Aqueous HCl (32 w %, 42.7 g) is added to a suspension of aniline-4-sulfonic acid (26 g) in water (200 mL) at 5 to 10° C., followed by addition of sodium nitrite (4 N, 38 mL) within 40 minutes. The reaction mixture is stirred for 1 hour, and then unreacted nitrite is destroyed by addition of sulfamic acid. A suspension containing the diazonium ion 8c (A¹ is 4-sulfophenyl) is obtained.

This suspension is added to a suspension of the aniline derivative 6a (24.2 g) in water (300 mL) at pH 2.0 to 2.5 within 30 minutes. The reaction mixture is stirred at pH 2.0 to 2.5 until the reaction was complete. The resulting suspension is filtered and the filter cake is dried in vacuo to yield 35.4 g of the coupling product 9c (A¹ is 4-sulfophenyl, R^{1B} is 2-hydroxyethyl, R² is hydrogen).

A solution of 4-nitrobenzoylchloride (6 g) in acetone (30 mL) is added to a suspension of the coupling product 9c (10 g) in water (150 g) at below 32° C. and at pH 6.5 to 7.0. The reaction mixture is stirred overnight, filtered and the filter cake is dried in vacuo to yield 11.1 g of the nitro compound 11c (A¹ is 4-sulfophenyl, R^{1B} is 2-hydroxyethyl, R², R³ and R⁴ are hydrogen).

Aqueous sodium sulfide (60 w %, 4.7 g) is added to a suspension of the nitro compound 11c (10 g) in water (100 g) at 50° C. The reaction mixture is stirred at 50 to 55° C. for 1 hour, then treated with sodium chloride. The resulting suspension is filtered and the filter cake is dried in vacuo to yield 9 g of the 4-amino-4′-azobenzanilide derivative of the formula 2bj.

### Examples 63 to 122

### Preparation of the 4-amino-4′-azobenzanilide derivative of the formula

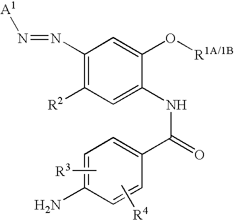

### (2A, respectively, 2B)

in which R³ and R⁴ are hydrogen, and

TABLE 2

| Example No/ | | | |
|---|---|---|---|
| Compound No | A¹ | R^{1A}, respectively, R^{1B} | R² |
| 63/2bk | 4-sulfophenyl | 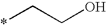 | hydrogen |
| 64/2b1 | 4-sulfophenyl | 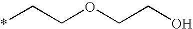 | hydrogen |
| 65/2bm | 4-sulfophenyl | 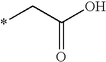 | hydrogen |
| 66/2bn | 4-sulfophenyl | 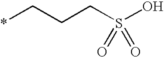 | hydrogen |
| 67/2bo | 4-sulfophenyl | 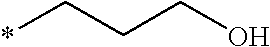 | hydrogen |
| 68/2bp | 4-sulfophenyl | 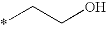 | methyl |
| 69/2bq | 4-sulfophenyl | 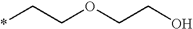 | methyl |
| 70/2br | 4-sulfophenyl | 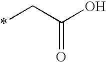 | methyl |
| 71/2bs | 4-sulfophenyl | 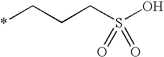 | methyl |
| 72/2bt | 4-sulfophenyl | 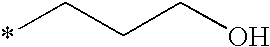 | methyl |
| 73/2bu | 3-sulfophenyl | 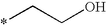 | hydrogen |
| 74/2bv | 3-sulfophenyl | 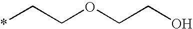 | hydrogen |
| 75/2bw | 3-sulfophenyl | 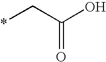 | hydrogen |
| 76/2bx | 3-sulfophenyl | 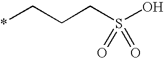 | hydrogen |
| 77/2by | 3-sulfophenyl | 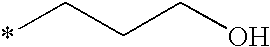 | hydrogen |
| 78/2bz | 3-sulfophenyl | 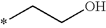 | methyl |
| 79/2ca | 3-sulfophenyl | 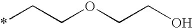 | methyl |
| 80/2cb | 3-sulfophenyl | 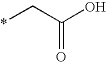 | methyl |
| 81/2cc | 3-sulfophenyl | 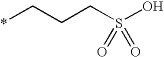 | methyl |
| 82/2cd | 3-sulfophenyl | 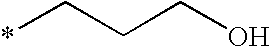 | methyl |
| 83/2ce | 4-sulfo-o-tolyl | 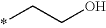 | hydrogen |
| 84/2cf | 4-sulfo-o-tolyl | 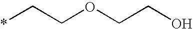 | hydrogen |
| 85/2cg | 4-sulfo-o-tolyl | 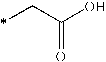 | hydrogen |
| 86/2ch | 4-sulfo-o-tolyl | 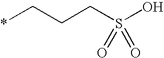 | hydrogen |
| 87/2ci | 4-sulfo-o-tolyl | 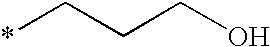 | hydrogen |
| 88/2cj | 4-sulfo-o-tolyl | 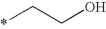 | methyl |
| 89/2ck | 4-sulfo-o-tolyl | 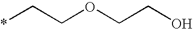 | methyl |
| 90/2cl | 4-sulfo-o-tolyl | 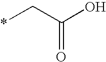 | methyl |
| 91/2cm | 4-sulfo-o-tolyl | 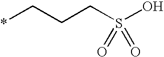 | methyl |
| 92/2cn | 4-sulfo-o-tolyl | 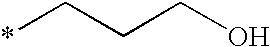 | methyl |
| 93/2co | 2,5-disulfophenyl | 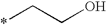 | hydrogen |
| 94/2cp | 2,5-disulfophenyl | 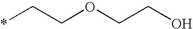 | hydrogen |
| 95/2cq | 2,5-disulfophenyl | 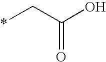 | hydrogen |
| 96/2cr | 2,5-disulfophenyl | 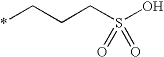 | hydrogen |
| 97/2cs | 2,5-disulfophenyl | 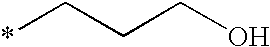 | hydrogen |
| 98/2ct | 2,5-disulfophenyl | 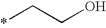 | methyl |
| 99/2cu | 2,5-disulfophenyl | 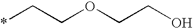 | methyl |
| 100/2cv | 2,5-disulfophenyl | 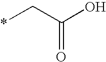 | methyl |
| 101/2cw | 2,5-disulfophenyl | 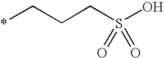 | methyl |
| 102/2cx | 2,5-disulfophenyl | 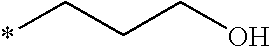 | methyl |
| 103/2cy | 3-sulfo-p-tolyl | 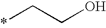 | hydrogen |
| 104/2cz | 3-sulfo-p-tolyl | 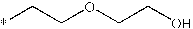 | hydrogen |
| 105/2da | 3-sulfo-p-tolyl | 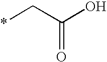 | hydrogen |
| 106/2db | 3-sulfo-p-tolyl | 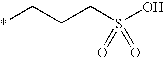 | hydrogen |
| 107/2dc | 3-sulfo-p-tolyl | 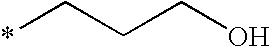 | hydrogen |
| 108/2dd | 3-sulfo-p-tolyl | 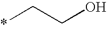 | methyl |
| 109/2de | 3-sulfo-p-tolyl | 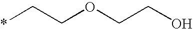 | methyl |
| 110/2df | 3-sulfo-p-tolyl | 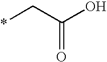 | methyl |
| 111/2dg | 3-sulfo-p-tolyl | 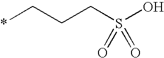 | methyl |
| 112/2dh | 3-sulfo-p-tolyl | 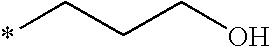 | methyl |
| 113/2di | 2-methoxy-5-sulfo-phenyl | 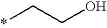 | hydrogen |
| 114/2dj | 2-methoxy-5-sulfo-phenyl | 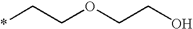 | hydrogen |
| 115/2dk | 2-methoxy-5-sulfo-phenyl | 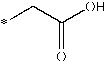 | hydrogen |
| 116/2dl | 2-methoxy-5-sulfo-phenyl | 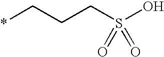 | hydrogen |
| 117/2dm | 2-methoxy-5-sulfo-phenyl | 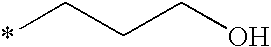 | hydrogen |
| 118/2dn | 2-methoxy-5-sulfo-phenyl | 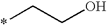 | methyl |
| 119/2do | 2-methoxy-5-sulfo-phenyl | 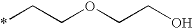 | methyl |
| 120/2dp | 2-methoxy-5-sulfo-phenyl | 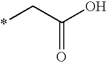 | methyl |
| 121/2dq | 2-methoxy-5-sulfo-phenyl | 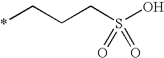 | methyl |
| 122/2dr | 2-methoxy-5-sulfo-phenyl | 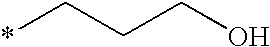 | methyl |

These 4-amino-4′-azobenzanilide derivatives are prepared in analogy to example 62.

### Example 123

### Preparation of the 4-amino-4′-azobenzanilide derivative of the formula

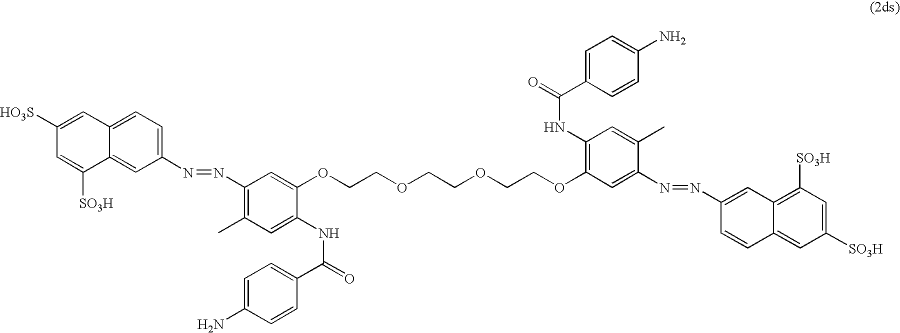

### (A¹ is 6,8-disulfo-2-naphthyl, R² is methyl, R³ and R⁴ are hydrogen and R^{1A}, respectively, R^{1C} is

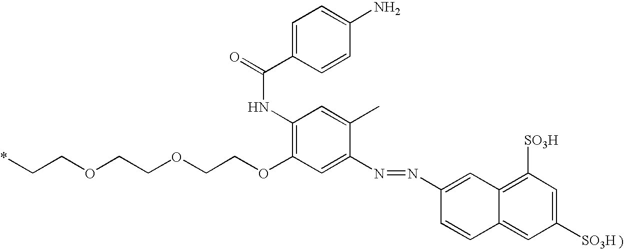

1,2-Bis(2-chloroethoxy)ethane (56.1 g) is added to a solution of 4-methyl-2-nitrophenol (91.8 g), potassium carbonate (91.2 g) and potassium iodide (12.4 g) in dimethylformamide (500 mL) at 70° C. within 40 minutes. The reaction mixture is stirred at 100° C. for 3 hours. Then it is cooled to 40° C. and filtered. The filtrate is concentrated in vacuo. The remaining oil is diluted with tert-butyl methyl ether and cooled to room temperature. A precipitate is obtained which is separated by filtration and dried to yield 92.2 g of the nitrobenzol derivative 13a (R² is methyl, X is CH₂CH₂OCH₂CH₂OCH₂CH₂).

Aqueous sodium sulfide (60 w %, 52 g) is added to a solution of the nitrobenzol derivative 13a (84.1 g) in dimethylformamide (250 mL) at 80° C. and the reaction mixture is stirred at 100° C. for 1 hour. The reaction mixture is cooled to room temperature and concentrated. The obtained suspension is filtered and the filter cake is dried in vacuo to yield 70.5 g of the aniline derivative 14a (R² is methyl, X is CH₂CH₂OCH₂CH₂OCH₂CH₂).

Aqueous HCl (32 w %, 18.8 g) is added to a suspension of 2-naphthylamine-6,8-disulfonic acid (20 g) in water (200 mL) at 5 to 10° C., followed by addition of sodium nitrite (4 N, 17 mL) within 40 minutes. The reaction mixture is stirred for 1 hour, and then unreacted nitrite is destroyed by addition of sulfamic acid. A suspension containing the diazonium ion 8a (A¹ is 6,8-disulfonaphthyl) is obtained.

This suspension is added to a suspension of the aniline derivative 14a (11.9 g) in water (150 mL) at pH 2.0 to 2.5 within 30 minutes. The reaction mixture is stirred at pH 2.0 to 4.0 until the reaction is complete. The reaction mixture is treated with sodium chloride, the resulting suspension is filtered and the filter cake is dried in vacuo to yield 24.5 g of the coupling product 15a (A¹ is 6,8-disulfonaphthyl, R² is methyl, X is CH₂CH₂OCH₂CH₂—OCH₂CH₂).

A solution of 4-nitrobenzoylchloride (9.7 g) in acetone (30 mL) is added to a suspension of the coupling product 15a (11.8 g) in water (100 g) at below 32° C. and at pH 6.5 to 7.0. The reaction mixture is stirred overnight, filtered and the filter cake is dried in vacuo to yield 10.8 g of the nitro compound 16a (A¹ is 6,8-disulfonaphthyl, R² is methyl, X is CH₂CH₂OCH₂CH₂OCH₂CH₂, R³ and R⁴ are hydrogen).

Aqueous sodium sulfide (60 w %, 4.9 g) is added to a suspension of the nitro compound 16a (10 g) in brine (20 w %, 100 g) at 50° C. The reaction mixture is stirred at 50 to 55° C. for 1 hour, cooled to room temperature and treated with sodium chloride. The resulting suspension is filtered and the filter cake is dried in vacuo to yield 6.4 g of the 4-amino-4′-azobenzanilide derivative of the formula 2ds.

### Examples 124 to 146

### Preparation of the 4-amino-4′-azobenzanilide derivative of the formula

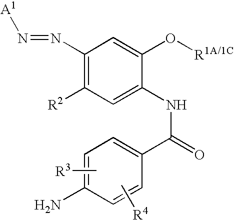

### (2A, respectively, 2C)

in which R^{1A}, respectively, R^{1C} is

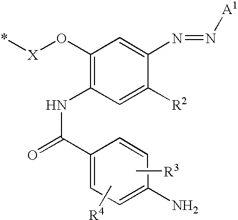

### X is CH₂CH₂OCH₂CH₂OCH₂CH₂, R³ and R⁴ are hydrogen, and

TABLE 3

| Example No/ | | |
|---|---|---|
| Compound No | A¹ | R² |
| 124/2dt | 6,8-disulfo-2-naphthyl | hydrogen |
| 125/2du | 4,8-disulfo-2-naphthyl | methyl |
| 126/2dv | 4,8-disulfo-2-naphthyl | hydrogen |
| 127/2dw | 3,6-disulfo-2-naphthyl | methyl |
| 128/2dx | 3,6-disulfo-2-naphthyl | hydrogen |
| 129/2dy | 5,7-disulfo-2-naphthyl | methyl |
| 130/2dz | 5,7-disulfo-2-naphthyl | hydrogen |
| 131/2ea | 1,5-disulfo-2-naphthyl | methyl |
| 132/2eb | 1,5-disulfo-2-naphthyl | hydrogen |
| 133/2ec | 1,6-disulfo-2-naphthyl | methyl |
| 134/2ed | 1,6-disulfo-2-naphthyl | hydrogen |
| 135/2ee | 4-sulfophenyl | hydrogen |
| 136/2ef | 4-sulfophenyl | methyl |
| 137/2eg | 3-sulfophenyl | hydrogen |
| 138/2eh | 3-sulfophenyl | methyl |
| 139/2ei | 4-sulfo-o-tolyl | hydrogen |
| 140/2ej | 4-sulfo-o-tolyl | methyl |
| 141/2ek | 2,5-disulfophenyl | hydrogen |
| 142/2el | 2,5-disulfophenyl | methyl |
| 143/2em | 3-sulfo-p-tolyl | hydrogen |
| 144/2en | 3-sulfo-p-tolyl | methyl |
| 145/2eo | 2-methoxy-5-sulfo- | hydrogen |
| | phenyl | |
| 146/2ep | 2-methoxy-5-sulfo- | methyl |
| | phenyl | |

These 4-amino-4′-azobenzanilide derivatives are prepared in analogy to example 123.

### Examples 147 to 170

### Preparation of the 4-amino-4′-azobenzanilide derivative of the formula

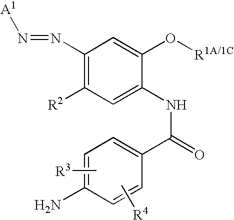

### (2A, respectively, 2C)

in which R^{1A} respectively, R^{1C} is

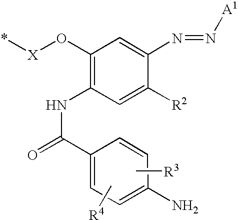

### X is CH₂CH₂CH₂CH₂CH₂CH₂, R³ and R⁴ are hydrogen, and

TABLE 4

| Example No/ | | |
|---|---|---|
| Compound No | A¹ | R² |
| 147/2eq | 6,8-disulfo-2-naphthyl | methyl |
| 148/2er | 6,8-disulfo-2-naphthyl | hydrogen |
| 149/2es | 4,8-disulfo-2-naphthyl | methyl |
| 150/2et | 4,8-disulfo-2-naphthyl | hydrogen |
| 151/2eu | 3,6-disulfo-2-naphthyl | methyl |
| 152/2ev | 3,6-disulfo-2-naphthyl | hydrogen |
| 153/2ew | 5,7-disulfo-2-naphthyl | methyl |
| 154/2ex | 5,7-disulfo-2-naphthyl | hydrogen |
| 155/2ey | 1,5-disulfo-2-naphthyl | methyl |
| 156/2ez | 1,5-disulfo-2-naphthyl | hydrogen |
| 157/2fa | 1,6-disulfo-2-naphthyl | methyl |
| 158/2fb | 1,6-disulfo-2-naphthyl | hydrogen |
| 159/2fc | 4-sulfophenyl | hydrogen |
| 160/2fd | 4-sulfophenyl | methyl |
| 161/2fe | 3-sulfophenyl | hydrogen |
| 162/2ff | 3-sulfophenyl | methyl |
| 163/2fg | 4-sulfo-o-tolyl | hydrogen |
| 164/2fh | 4-sulfo-o-tolyl | methyl |
| 165/2fi | 2,5-disulfophenyl | hydrogen |
| 166/2fj | 2,5-disulfophenyl | methyl |
| 167/2fk | 3-sulfo-p-tolyl | hydrogen |
| 168/2fl | 3-sulfo-p-tolyl | methyl |
| 169/2fm | 2-methoxy-5-sulfo- | hydrogen |
| | phenyl | |
| 170/2fn | 2-methoxy-5-sulfo- | methyl |
| | phenyl | |

These 4-amino-4′-azobenzanilide derivatives are prepared in analogy to example 123.

### Example 171

### Preparation of the 4,4′-diazobenzanilide derivative of the formula

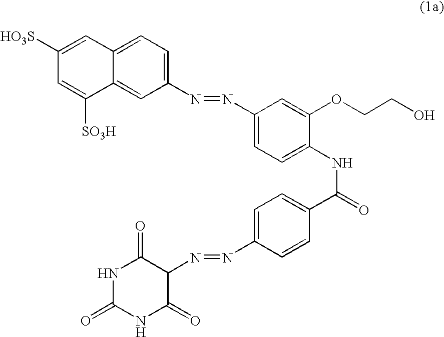

### (A¹ is 6,8-disulfo-2-naphthyl, R^{1A} is 2-hydroxyethyl, R², R³ and R⁴ are hydrogen, A² is

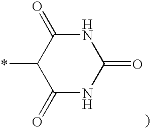

Aqueous sodium nitrite (4 N, 3 mL) is added to a suspension of the 4-amino-4′-azobenzanilide derivative 2a (7 g), which is prepared as described in example 1, in water (100 g). The obtained suspension is cooled to 0 to 5° C. and added to a solution of HCl (32 w %, 4 g) in brine (25 w %, 70 g) at 5° C. within 40 minutes. The reaction mixture is stirred for 1 h. Then unreacted nitrite is destroyed by addition of sulfamic acid. A suspension containing the diazonium ion 17a (A¹ is 6,8-disulfo-2-naphthyl, R^{1A} is 2-hydroxyethyl, R², R³ and R⁴ are hydrogen) is obtained.

Barbituric acid (1.55 g) is added to this suspension. The pH of the reaction mixture is adjusted to 4.0. The reaction mixture is warmed to room temperature at pH 3.5 to 4.0, and stirred until the reaction is complete. The resulting suspension is filtered and the filter cake is dried in vacuo to yield the 5.5 g of the 4,4′-diazobenzanilide derivative 1a.

### Example 172

### Preparation of the 4,4′-diazobenzanilide derivative of the formula

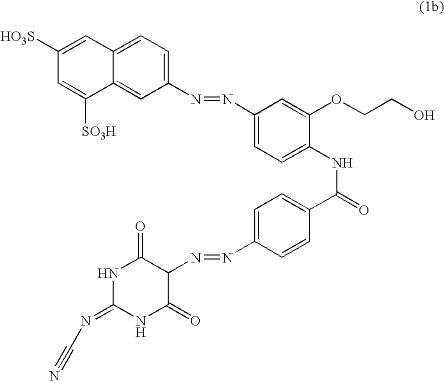

### (A¹ is 6,8-disulfo-2-naphthyl, R^{1A} is 2-hydroxyethyl, R², R³ and R⁴ are hydrogen, A² is

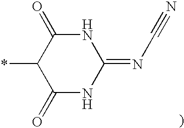

Aqueous sodium nitrite (4 N, 3 mL) is added to a suspension of the 4-amino-4′-azobenzanilide derivative 2a (7 g), which is prepared as described in example 1, in water (100 g). The obtained suspension is cooled to 0 to 5° C. and added to a solution of HCl (32 w %, 3.5 g) in brine (25 w %, 70 g) at 5° C. within 40 minutes. The reaction mixture is stirred for 1 h. Then unreacted nitrite is destroyed by addition of sulfamic acid. A suspension containing the diazonium ion 17a (A¹ is 6,8-disulfo-2-naphthyl, R^{1A} is 2-hydroxyethyl, R², R³ and R⁴ are hydrogen) is obtained.

Cyanoiminobarbituric acid (1.85 g) is added to this suspension. The pH of the reaction mixture is adjusted to 4.0. The reaction mixture is warmed to room temperature at pH 3.5 to 4.0, and stirred until the reaction was complete. The resulting suspension is filtered and the filter cake is dried in vacuo to yield the 7.2 g of the 4,4′-diazobenzanilide derivative 1b.

### Example 173

### Preparation of the 4,4′-diazobenzanilide derivative of the formula

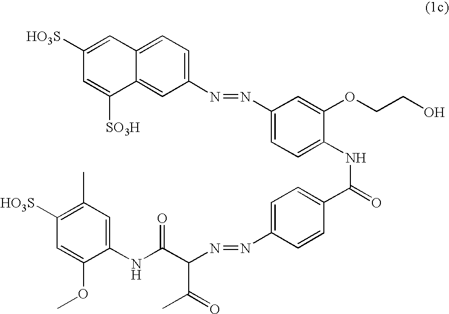

### (A¹ is 6,8-disulfo-2-naphthyl, R^{1A} is 2-hydroxyethyl, R², R³ and R⁴ are hydrogen, A² is

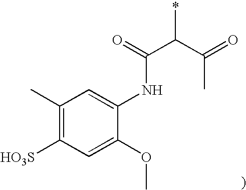

Aqueous sodium nitrite (4 N, 3 mL) is added to a suspension of the 4-amino-4′-azobenzanilide derivative 2a (7 g), which is prepared as described in example 1, in water (100 g). The obtained suspension is cooled to 0 to 5° C. and added to a solution of HCl (32 w %, 3.5 g) in brine (25 w %, 70 g) at 5° C. within 40 minutes. The reaction mixture is stirred for 1 h. Then unreacted nitrite is destroyed by addition of sulfamic acid. A suspension containing the diazonium ion 17a (A¹ is 6,8-disulfo-2-naphthyl, R^{1A} is 2-hydroxyethyl, R², R³ and R⁴ are hydrogen) is obtained.

2-Methoxy-5-methyl-4-sulfoacetoacetanilide, sodium salt (3.9 g) is added to this suspension. The pH of the reaction mixture is adjusted to 4.0. The reaction mixture is warmed to room temperature at pH 3.5 to 4.0, and stirred until the reaction was complete. The resulting suspension was filtered and the filter cake was dried in vacuo to yield the 8.3 g of the 4,4′-diazobenzanilide derivative 1c.

### Example 174

### Preparation of the 4,4′-diazobenzanilide derivative of the formula

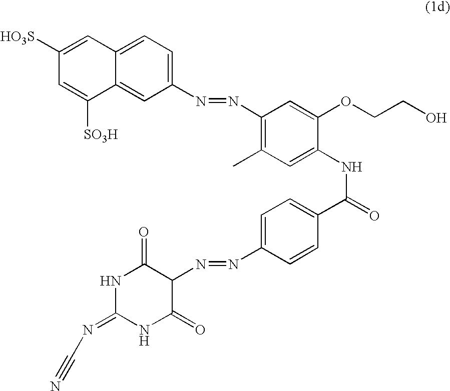

### (A¹ is 6,8-disulfo-2-naphthyl, R^{1A} is 2-hydroxyethyl, R² is methyl, R³ and R⁴ are hydrogen, A² is

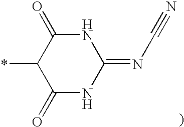

Sodium chloride (20 g) and HCl (32 w %, 3.5 g) are added to a suspension of the 4-amino-4′ azobenzanilide derivative 2f (7 g), which is prepared in analogy to example 1, in water (100 g). The obtained suspension is cooled to 0 to 5° C. and aqueous sodium nitrite (4 N, 3 mL) are added at 0 to 5° C. within 40 minutes. The reaction mixture is stirred for 1 h. Then unreacted nitrite is destroyed by addition of sulfamic acid. A suspension containing the diazonium ion 17b (A¹ is 6,8-disulfo-2-naphthyl, R^{1A} is 2-hydroxyethyl, R² is methyl, R³ and R⁴ are hydrogen) is obtained.

Cyanoiminobarbituric acid (1.81 g) is added to this suspension. The pH of the reaction mixture is adjusted to 4.0. The reaction mixture is warmed to room temperature at pH 3.5 to 4.0, and stirred until the reaction is complete. The resulting suspension is filtered and the filter cake is dried in vacuo to yield the 8.9 g of the 4,4′-diazobenzanilide derivative 1d.

### Example 175

### Preparation of the 4,4′-diazobenzanilide derivative of the formula

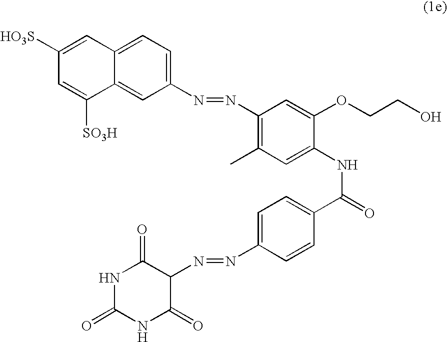

### (A¹ is 6,8-disulfo-2-naphthyl, R^{1A} is 2-hydroxyethyl, R² is methyl, R³ and R⁴ are hydrogen, A² is

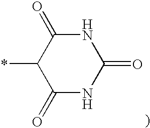

Sodium chloride (20 g) and HCl (32 w %, 3.5 g) are added to a suspension of the 4-amino-4′-azobenzanilide derivative 2f (7 g), which is prepared in analogy to example 1, in water (100 g). The obtained suspension is cooled to 0 to 5° C. and aqueous sodium nitrite (4 N, 3 mL) is added at 0 to 5° C. within 40 minutes. The reaction mixture is stirred for 1 h. Then unreacted nitrite is destroyed by addition of sulfamic acid. A suspension containing the diazonium ion 17b (A¹ is 6,8-disulfo-2-naphthyl, R^{1A} is 2-hydroxyethyl, R² is methyl, R³ and R⁴ are hydrogen) is obtained.

Barbituric acid (1.53 g) is added to this suspension. The pH of the reaction mixture is adjusted to 4.0. The reaction mixture is warmed to room temperature at pH 3.5 to 4.0, and stirred until the reaction is complete. The resulting suspension is filtered and the filter cake is dried in vacuo to yield the 6.5 g of the 4,4′-diazobenzanilide derivative 1e.

### Example 176

### Preparation of the 4,4′-diazobenzanilide derivative of the formula

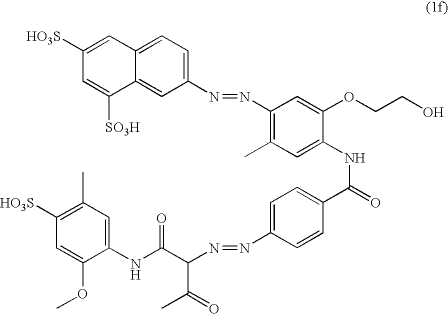

### (A¹ is 6,8-disulfo-2-naphthyl, R^{1A} is 2-hydroxyethyl, R² is methyl, R³ and R⁴ are hydrogen, A² is

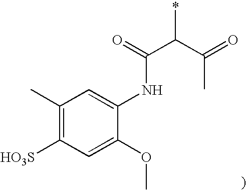

Sodium chloride (20 g) and HCl (32 w %, 3.5 g) are added to a suspension of the 4-amino-4′-azobenzanilide derivative 2f (7 g), which is prepared in analogy to example 1, in water (100 g). The obtained suspension is cooled to 0 to 5° C. and aqueous sodium nitrite (4 N, 3 mL) is added at 0 to 5° C. within 40 minutes. The reaction mixture is stirred for 1 h. Then unreacted nitrite is destroyed by addition of sulfamic acid. A suspension containing the diazonium ion 17b (A¹ is 6,8-disulfo-2-naphthyl, R^{1A} is 2-hydroxyethyl, R² is methyl, R³ and R⁴ are hydrogen) is obtained.

2-Methoxy-5-methyl-4-sulfoacetoacetanilide, sodium salt (3.84 g) is added to this suspension. The pH of the reaction mixture is adjusted to 6.5. The reaction mixture is warmed to room temperature at pH 6.5 to 7.0, and stirred until the reaction is complete. The resulting suspension is filtered and the filter cake is dried in vacuo to yield 10 g of the 4,4′-diazobenzanilide derivative 1f.

### Example 177

### Preparation of the 4,4′-diazobenzanilide derivative of the formula

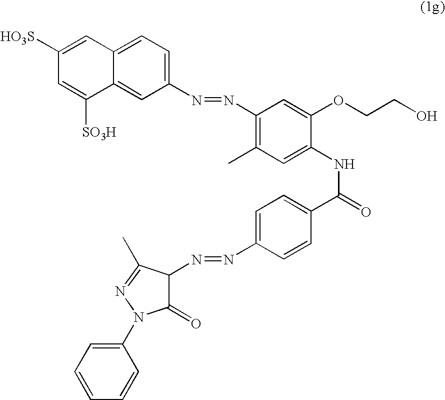

### (A¹ is 6,8-disulfo-2-naphthyl, R^{1A} is 2-hydroxyethyl, R² is methyl, R³ and R⁴ are hydrogen, A² is

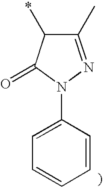

Sodium chloride (15 g) and HCl (32 w %, 2 g) are added to a suspension of the 4-amino-4′-azobenzanilide derivative 2f (3.6 g), which is prepared in analogy to example 1, in water (75 g). The obtained suspension is cooled to 0 to 5° C. and aqueous sodium nitrite (4 N, 1.5 mL) is added at 0 to 5° C. within 40 minutes. The reaction mixture is stirred for 1 h. Then unreacted nitrite is destroyed by addition of sulfamic acid. A suspension containing the diazonium ion 17b (A¹ is 6,8-disulfo-2-naphthyl, R^{1A} is 2-hydroxyethyl, R² is methyl, R³ and R⁴ are hydrogen) is obtained.

3-Methyl-1-phenyl-2-pyrazolin-5-one (1.07 g) is added to this suspension. The pH of the reaction mixture is adjusted to 5.0. The reaction mixture is warmed to room temperature at pH 5.0 to 5.5, and stirred until the reaction was complete. The resulting suspension is filtered and the filter cake is dried in vacuo to yield 4.5 g of the 4,4′-diazobenzanilide derivative 1 g.

### Example 178

### Preparation of the 4,4′-diazobenzanilide derivative of the formula

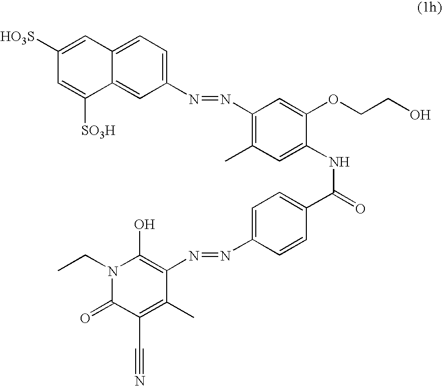

### (A¹ is 6,8-disulfo-2-naphthyl, R^{1A} is 2-hydroxyethyl, R² is methyl, R³ and R⁴ are hydrogen, A² is

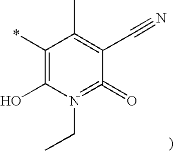

Sodium chloride (20 g) and HCl (32 w %, 2.5 g) are added to a suspension of the 4-amino-4′-azobenzanilide derivative 2f (4.5 g), which is prepared in analogy to example 1, in water (100 g). The obtained suspension is cooled to 0 to 5° C. and aqueous sodium nitrite (4 N, 2 mL) is added at 0 to 5° C. within 40 minutes. The reaction mixture is stirred for 1 h. Then unreacted nitrite is destroyed by addition of sulfamic acid. A suspension containing the diazonium ion 17b (A¹ is 6,8-disulfo-2-naphthyl, R^{1A} is 2-hydroxyethyl, R² is methyl, R³ and R⁴ are hydrogen) is obtained.

3-Cyano-1-ethyl-6-hydroxy-4-methyl-2-pyridone (1.35 g) is added to this suspension. The pH of the reaction mixture is adjusted to 3.0. The reaction mixture is warmed to room temperature at pH 3.0 to 3.5, and stirred until the reaction is complete. The resulting suspension is filtered and the filter cake is dried in vacuo to yield 5.2 g of the 4,4′-diazobenzanilide derivative 1 h.

### Examples 179 to 196

### Preparation of a 4,4′-diazobenzanilide derivative of the formula

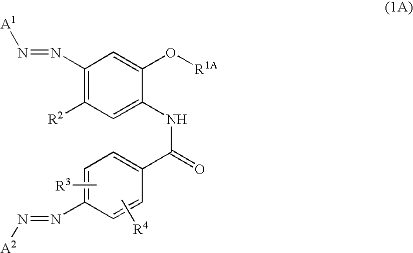

in which A¹ is 6,8-disulfo-2-naphthyl, R^{1A} is 2-hydroxyethyl, R³ and R⁴ are hydrogen, and

TABLE 5

| Example No/ | | |
|---|---|---|
| Compound No | A² | R² |
| 179/1i | 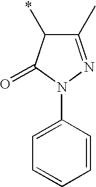 | hydrogen |
| 180/1j | 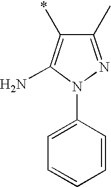 | hydrogen |
| 181/1k | 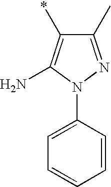 | methyl |
| 182/1l | 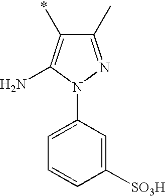 | hydrogen |
| 183/1m | 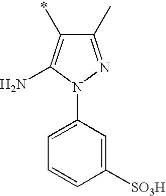 | methyl |
| 184/1n | 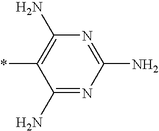 | hydrogen |
| 185/1o | 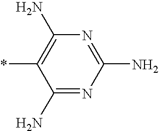 | methyl |
| 186/1p | 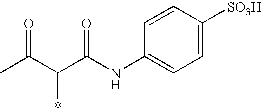 | hydrogen |
| 187/1q | 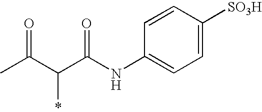 | methyl |
| 188/1r | 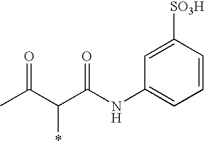 | hydrogen |
| 189/1s | 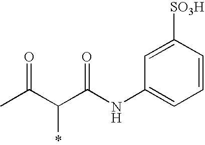 | methyl |
| 190/1t | 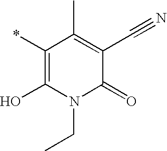 | hydrogen |
| 191/1u | 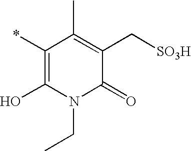 | hydrogen |
| 192/1v | 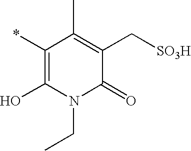 | methyl |
| 193/1w | 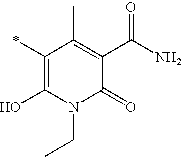 | hydrogen |
| 194/1x | 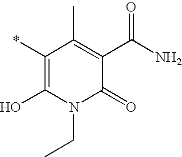 | methyl |
| 195/1y | 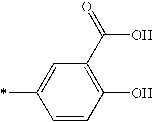 | hydrogen |
| 196/1z | 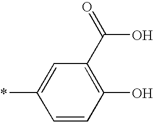 | methyl |

These 4,4′-diazobenzanilide derivatives are prepared in analogy to example 171.

### Example 197 to 200

### Preparation of a 4,4′-diazobenzanilide derivative of the formula

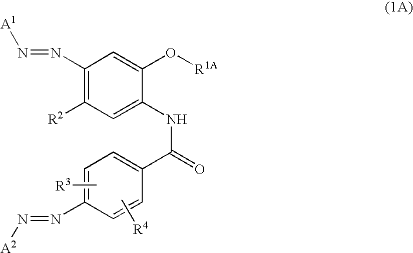

in which A¹ is 4,8-disulfo-2-naphthyl, R^{1A} is 2-hydroxyethyl, R³ and R⁴ are hydrogen, and

TABLE 6

| Example No/ | | |
|---|---|---|
| Compound No | A² | R² |
| 197/1aa | 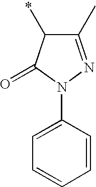 | hydrogen |
| 198/1ab | 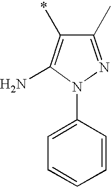 | hydrogen |
| 199/1ac | 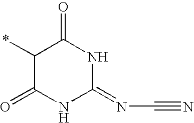 | hydrogen |
| 200/1ad | 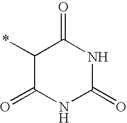 | hydrogen |
| 200/1ae | 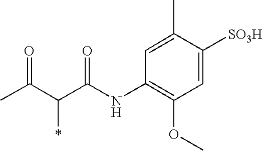 | hydrogen |

These 4,4′-diazobenzanilide derivatives are prepared in analogy to example 171 starting from 4-amino-4′-azobenzanilide derivative 2k (example 11).

### Examples 201 to 203

### Preparation of a 4,4′-diazobenzanilide derivative of the formula

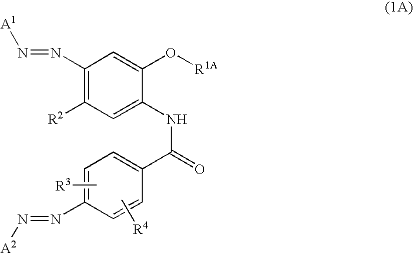

in which A¹ is 6-sulfo-2-naphthyl, R^{1A} is 2-hydroxyethyl, R³ and R⁴ are hydrogen, and

TABLE 7

| Example No/ | | |
|---|---|---|
| Compound No | A² | R² |
| 201/1af | 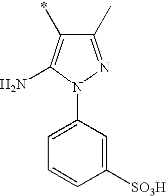 | methyl |
| 202/1ag | 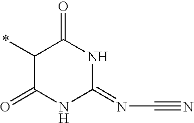 | methyl |
| 203/1ah | 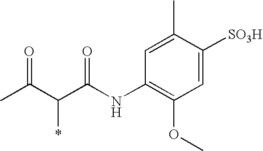 | methyl |

These 4,4′-diazobenzanilide derivatives are prepared in analogy to example 171 starting from 4-amino-4′-azobenzanilide derivative 1bi (example 61).

### Example 204

### Preparation the 4,4′-diazobenzanilide derivative of the formula

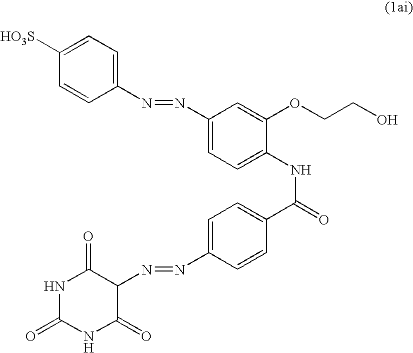

### (A¹ is 4-sulfophenyl, R^{1A} is 2-hydroxyethyl, R² is hydrogen, R³ and R⁴ are hydrogen, A² is

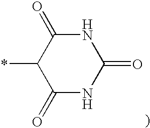

Aqueous sodium nitrite (4 N, 3 mL) is added to a suspension of the 4-amino-4′-azobenzanilide derivative 2bj (6 g), which is prepared as described in example 62, in water (100 g). The obtained suspension is cooled to 0 to 5° C. and added to a solution of HCl (32 w %, 4.5 g) and sodium chloride (25 g) in water (50 g) at 5° C. within 1 hour. The reaction mixture is stirred for 1 h. Then unreacted nitrite is destroyed by addition of sulfamic acid. A suspension containing the diazonium ion 17c (A¹ is 4-sulfophenyl, R^{1A} is 2-hydroxyethyl, R², R³ and R⁴ are hydrogen) is obtained.

Barbituric acid (1.72 g) is added to this suspension. The pH of the reaction mixture is adjusted to 4.0. The reaction mixture is warmed to room temperature at pH 4.0 to 4.5, and stirred until the reaction is complete. The resulting suspension is filtered and the filter cake is dried in vacuo to yield 7.5 g of the 4,4′-diazobenzanilide derivative 1ai.

### Example 205

### Preparation the 4,4′-diazobenzanilide derivative of the formula

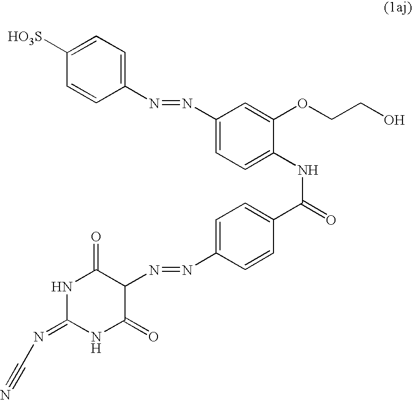

### (A¹ is 4-sulfophenyl, R^{1A} is 2-hydroxyethyl, R² is hydrogen, R³ and R⁴ are hydrogen, A² is

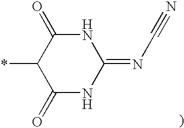

Aqueous sodium nitrite (4 N, 3 mL) is added to a suspension of the 4-amino-4′-azobenzanilide derivative 2bj (6 g), which is prepared as described in example 62, in water (100 g). The obtained suspension is cooled to 0 to 5° C. and added to a solution of HCl (32 w %, 4.5 g) and sodium chloride (25 g) in water (50 g) at 5° C. within 1 hour. The reaction mixture is stirred for 1 h. Then unreacted nitrite is destroyed by addition of sulfamic acid. A suspension containing the diazonium ion 17c (A¹ is 4-sulfophenyl, R^{1A} is 2-hydroxyethyl, R², R³ and R⁴ are hydrogen) is obtained.

Cyanoiminobarbituric acid (2.04 g) is added to this suspension. The pH of the reaction mixture is adjusted to 4.0. The reaction mixture is warmed to room temperature at pH 4.0 to 4.5, and stirred until the reaction is complete. The resulting suspension is filtered and the filter cake is dried in vacuo to yield 4.9 g of the 4,4′-diazobenzanilide derivative 1aj.

### Example 206

### Preparation the 4,4′-diazobenzanilide derivative of the formula

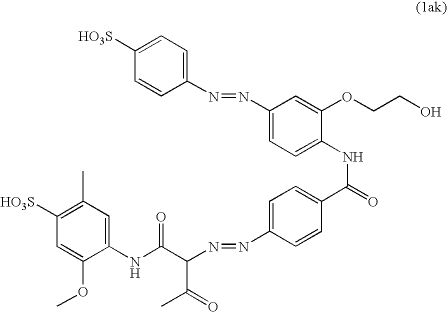

### (A¹ is 4-sulfophenyl, R^{1A} is 2-hydroxyethyl, R² is hydrogen, R³ and R⁴ are hydrogen, A² is

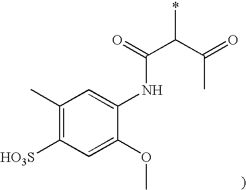

Aqueous sodium nitrite (4 N, 3 mL) is added to a suspension of the 4-amino-4′-azobenzanilide derivative 2bj (6 g), which is prepared as described in example 62, in water (100 g). The obtained suspension is cooled to 0 to 5° C. and added to a solution of HCl (32 w %, 4.5 g) and sodium chloride (25 g) in water (50 g) at 5° C. within 1 hour. The reaction mixture is stirred for 1 h. Then unreacted nitrite is destroyed by addition of sulfamic acid. A suspension containing the diazonium ion 17c (A¹ is 4-sulfophenyl, R^{1A} is 2-hydroxyethyl, R², R³ and R⁴ are hydrogen) is obtained.

2-Methoxy-5-methyl-4-sulfoacetoacetanilide, sodium salt (4.33 g) is added to this suspension. The pH of the reaction mixture is adjusted to 4.0. The reaction mixture is warmed to room temperature at pH 6.5 to 7.0, and stirred until the reaction is complete. The resulting suspension is filtered and the filter cake is dried in vacuo to yield 8.4 g of the 4,4′-diazobenzanilide derivative 1ak.

### Examples 207 to 229

### Preparation of a 4,4′-diazobenzanilide derivative of the formula

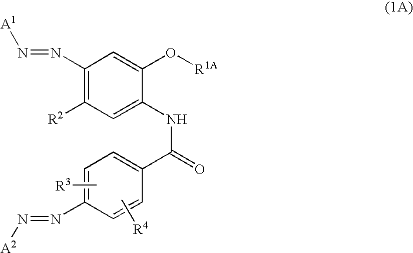

in which A¹ is 4-sulfophenyl, R^{1A} is 2-hydroxyethyl, R³ and R⁴ are hydrogen, and

TABLE 8

| Example No/ | | |
|---|---|---|
| Compound No | A² | R² |
| 207/1al | 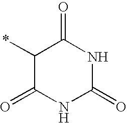 | methyl |
| 208/1am | 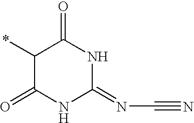 | methyl |
| 209/1an | 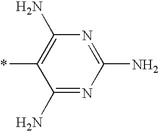 | hydrogen |
| 210/1ao | 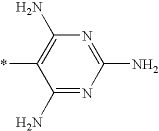 | methyl |
| 211/1ap | 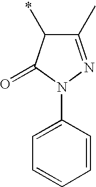 | hydrogen |
| 212/1aq | 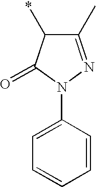 | methyl |
| 213/1ar | 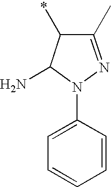 | hydrogen |
| 214/1as | 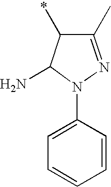 | methyl |
| 215/1at | 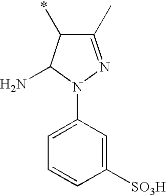 | hydrogen |
| 216/1au | 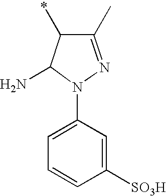 | methyl |
| 217/1av | 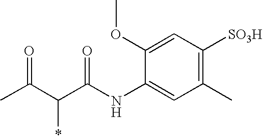 | methyl |
| 218/1aw | 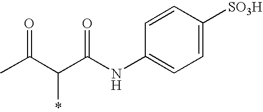 | hydrogen |
| 219/1ax | 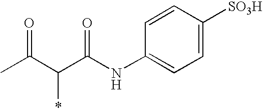 | methyl |
| 220/1ay | 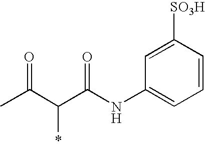 | hydrogen |
| 221/1az | 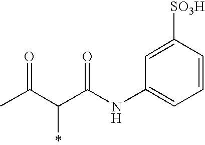 | methyl |
| 222/1ba | 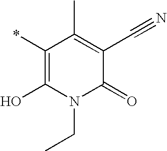 | hydrogen |
| 223/1bb | 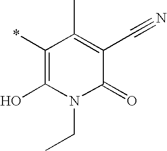 | methyl |
| 224/1bc | 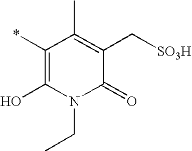 | hydrogen |
| 225/1bd | 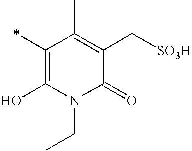 | methyl |
| 226/1be | 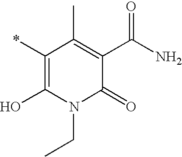 | hydrogen |
| 227/1bf | 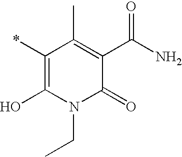 | methyl |
| 228/1bg | 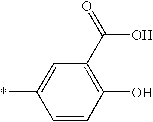 | hydrogen |
| 229/1bh | 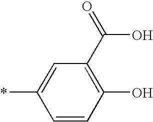 | methyl |

These 4,4′-diazobenzanilide derivatives are prepared in analogy to example 204.

### Example 230

### Preparation of the 4,4′-diazobenzanilide derivative of the formula

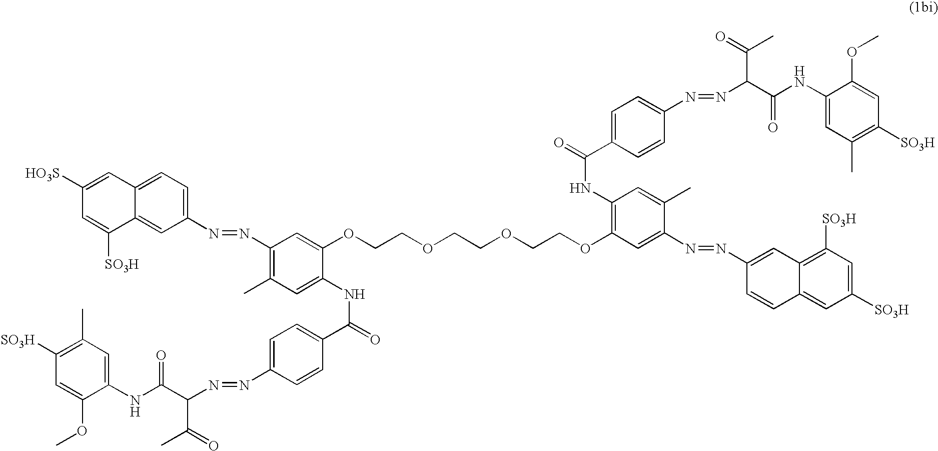

### (A¹ is 6,8-disulfo-2-naphthyl, R² is methyl, R³ and R⁴ are hydrogen, R^{1A} is

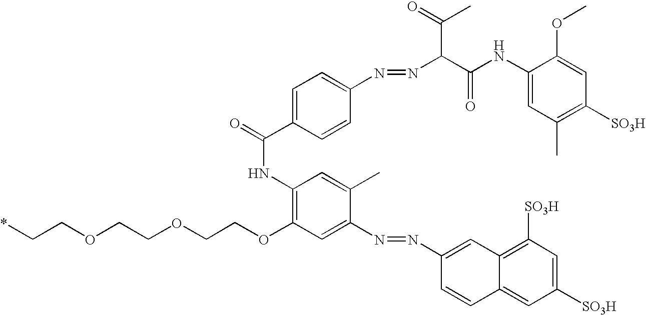

### A² is

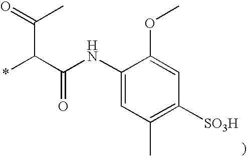

Aqueous sodium nitrite (4 N, 2.1 mL) is added to a suspension of the 4-amino-4′-azobenzanilide derivative 2ds (5 g), which is prepared as described in example 123, in water (100 g). The obtained suspension is cooled to 0 to 5° C. and added to a solution of HCl (32 w %, 2.8 g) and sodium chloride (20 g) in water (50 g) at 5° C. within 1 hour. The reaction mixture is stirred for 1 h. Then unreacted nitrite is destroyed by addition of sulfamic acid. A suspension obtaining the diazonium ion 17d (A¹ is 6,8-disulfo-2-naphthyl, R² is methyl, R³ and R⁴ are hydrogen, and R^{1A} is

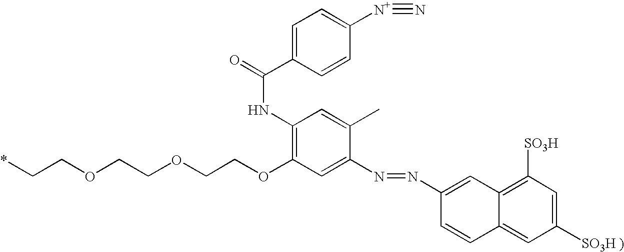

is obtained.

2-Methoxy-5-methyl-4-sulfoacetoacetanilide, sodium salt (2.71 g) is added to this suspension. The pH of the reaction mixture is adjusted to 4.0. The reaction mixture is warmed to room temperature at pH 6.5 to 7.0, and stirred until the reaction is complete. The resulting suspension is filtered and the filter cake is dried in vacuo to yield 4.5 g of the 4,4′-diazobenzanilide derivative 1bi.

### Example 231

### Preparation of the 4,4′-diazobenzanilide derivative of the formula

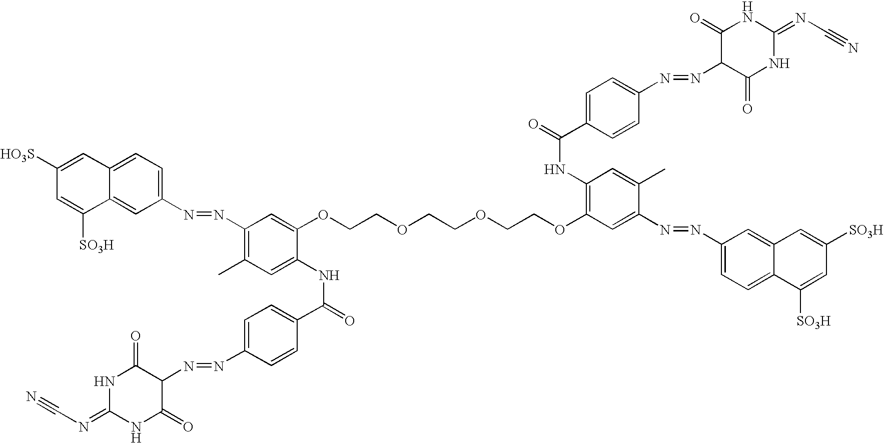

### (A¹ is 6,8-disulfo-2-naphthyl, R² is methyl, R³ and R⁴ are hydrogen, R^{1A} is

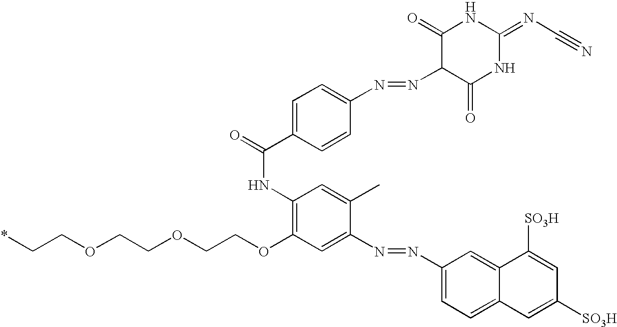

### A² is

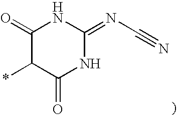

Aqueous sodium nitrite (4 N, 1.7 mL) is added to a suspension of the 4-amino-4′-azobenzanilide derivative 2ds (4 g), which is prepared as described in example 123, in water (100 g). The obtained suspension is cooled to 0 to 5° C. and added to a solution of HCl (32 w %, 2.0 g) and sodium chloride (20 g) in water (50 g) at 5° C. within 1 hour. The reaction mixture is stirred for 1 h. Then unreacted nitrite is destroyed by addition of sulfamic acid. A suspension containing the diazonium ion 17d (A¹ is 6,8-disulfo-2-naphthyl, R² is methyl, R³ and R⁴ are hydrogen, and R^{1A} is

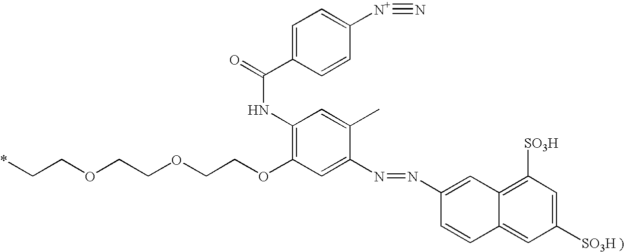

is obtained.

Cyanoiminobarbituric acid (1.01 g) is added to this suspension. The pH of the reaction mixture is adjusted to 4.0. The reaction mixture is warmed to room temperature at pH 4.5 to 5.0, and stirred until the reaction is complete. The resulting suspension is filtered and the filter cake is dried in vacuo to yield 2.9 g of the 4,4′-diazobenzanilide derivative 1bj.

### Examples 232 to 255

### Preparation of a 4,4′-diazobenzanilide derivative of the formula

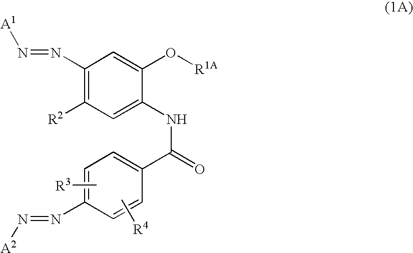

in which A¹ is 6,8-disulfo-2-naphthyl, R³ and R⁴ are hydrogen, and R^{1A} is

TABLE 9

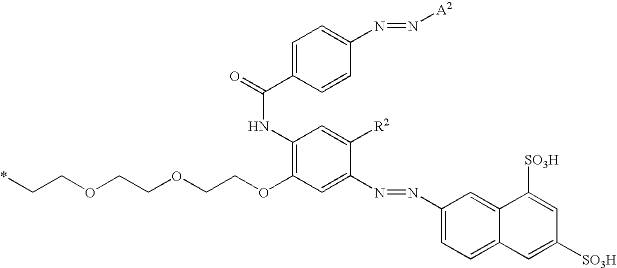

and

| Example No/Compound No | A² | R² |
|---|---|---|
| 232/1bk | 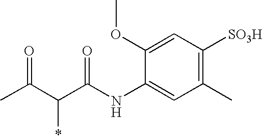 | hydrogen |
| 233/1bl | 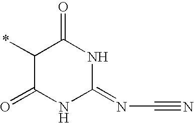 | hydrogen |
| 234/1bm | 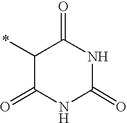 | hydrogen |
| 235/1bn | 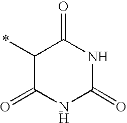 | methyl |
| 236/1bo | 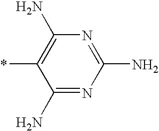 | hydrogen |
| 237/1bp | 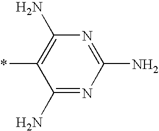 | methyl |
| 238/1bq | 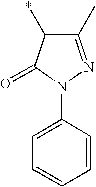 | hydrogen |
| 239/1br | 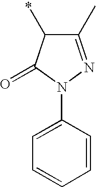 | methyl |
| 240/1bs | 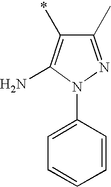 | hydrogen |
| 241/1bt | 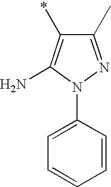 | methyl |
| 242/1bu | 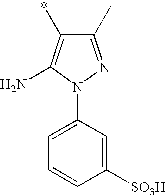 | hydrogen |
| 243/1bv | 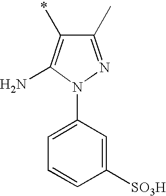 | methyl |
| 244/1bw | 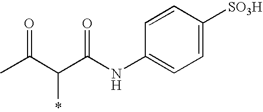 | hydrogen |
| 245/1bx | 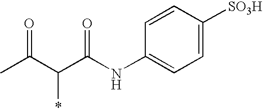 | methyl |
| 246/1by | 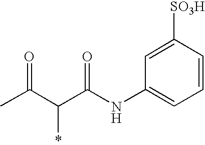 | hydrogen |
| 247/1bz | 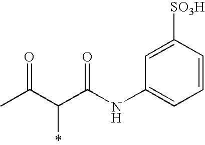 | methyl |
| 248/1ca | 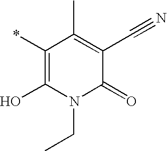 | hydrogen |
| 249/1cb | 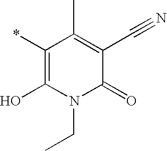 | methyl |
| 250/1cc | 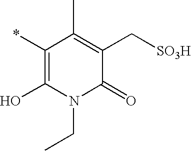 | hydrogen |
| 251/1cd | 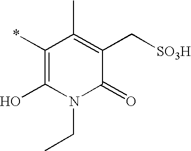 | methyl |
| 252/1ce | 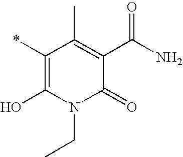 | hydrogen |
| 253/1cf | 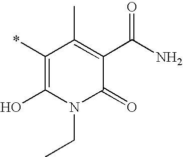 | methyl |
| 254/1cg | 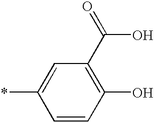 | hydrogen |
| 255/1ch | 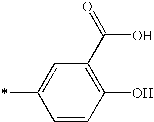 | methyl |

These 4,4′-diazobenzanilide derivatives are prepared in analogy to example 231.

### Examples 256 to 281

### Preparation of a 4,4′-diazobenzanilide derivative of the formula

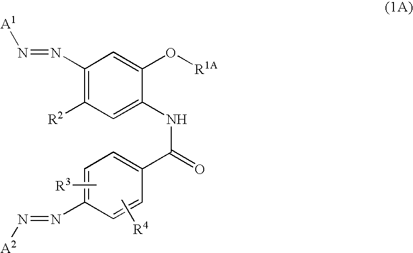

in which A¹ is 4-sulfophenyl, R³ and R⁴ are hydrogen, and R^{1A} is

TABLE 10

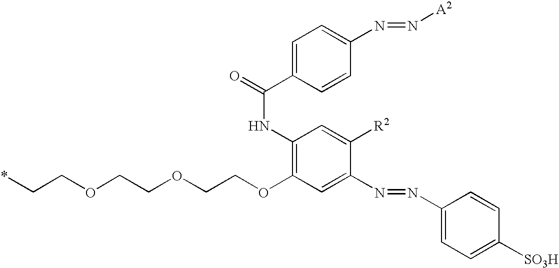

and

| Example No/Compound No | A² | R² |
|---|---|---|
| 256/1ci | 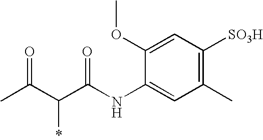 | hydrogen |
| 257/1cj | 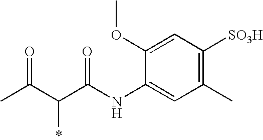 | methyl |
| 258/1ck | 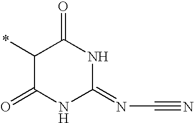 | hydrogen |
| 259/1cl | 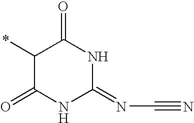 | methyl |
| 260/1cm | 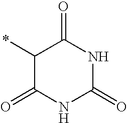 | hydrogen |
| 261/1cn | 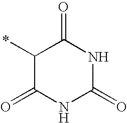 | methyl |
| 262/1co | 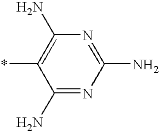 | hydrogen |
| 263/1cp | 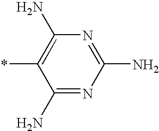 | methyl |
| 264/1cq | 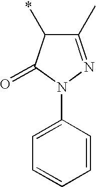 | hydrogen |
| 265/1cr | 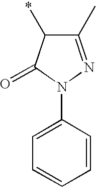 | methyl |
| 266/1cs | 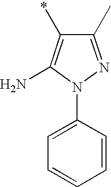 | hydrogen |
| 267/1ct | 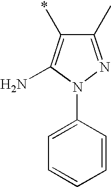 | methyl |
| 268/1cu | 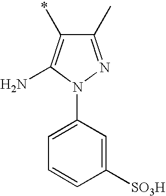 | hydrogen |
| 269/1cv | 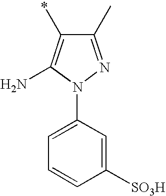 | methyl |
| 270/1cw | 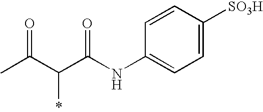 | hydrogen |
| 271/1cx | 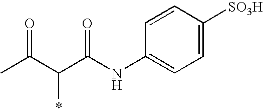 | methyl |
| 272/1cy | 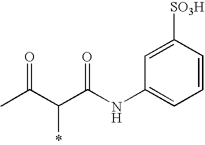 | hydrogen |
| 273/1cz | 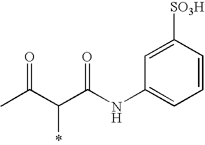 | methyl |
| 274/1da | 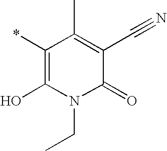 | hydrogen |
| 275/1db | 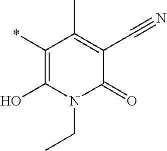 | methyl |
| 276/1dc | 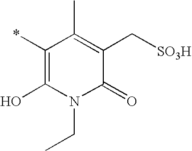 | hydrogen |
| 277/1dd | 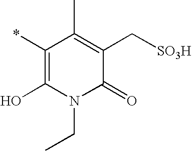 | methyl |
| 278/1de | 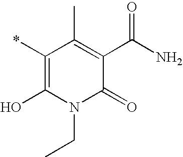 | hydrogen |
| 279/1df | 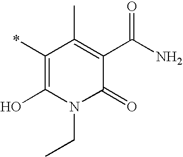 | methyl |
| 280/1dg | 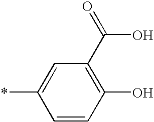 | hydrogen |
| 281/1dh | 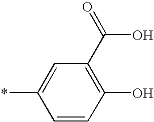 | methyl |

These 4,4′-diazobenzanilide derivatives are prepared in analogy to example 231.

### Application Examples

A fiber mixture of a suspension of 50% by weight sulfite long fiber bleached (spruce) and a suspension of 50% by weight sulfite short fiber bleached (beech) is suspended in deionised water, as a 2% suspension, refined and beaten to a degree of 22°SR (Schopper Riegler). After dewatering by means of a centrifuge and testing for dry weight, the equivalent to 10 g dry fiber is placed in a beaker and diluted with tab water to a final volume of 500 mL. After stirring for 1 hour, an amount of the respective 4,4′-diazobenzanilide derivative sufficient to produce a dyeing of 0.2 reference depth based on the weight of dry fibre, as a 5 g/L aqueous solution, is added to the furnish suspension and stirring is continued for further 15 minutes. The suspension is made up to 700 mL with tab water and from 300 mL of the resulting suspension a hand sheet is produced using a Lhomargy sheet former. After drying on a cylinder at 90° C. for 12 minutes, the CIELab coordinates and degrees of exhaustion of the dyes in the dyeings obtained are measured. The CIELab coordinates are used to calculate the shade of the dye (characterized by the °Hue value) and the brilliance of the dyeing (characterized by the C* value). The backwater ratings of the effluents are also assessed on a scale of from 1 to 5. The lighfastness is determined according to ISO/105/B02 using a xenon lamp and blue wool references corresponding to a scale from 1 to 8.

The results are summarized in Table 11 below.

TABLE 11

| | Amount Dye | | | | | |
|---|---|---|---|---|---|---|
| 4,4′-diazo- | [% dry | | | | Degree of | |
| benzanilide | weight/dry | | | Back- | Exhaustion | Light- |
| derivative | weight fiber] | °Hue | C* | water | [%] | fastness |
| 1a | 0.31 | 90.9 | 60.7 | 4+ | 93-95 | 4 |
| 1b | 0.3 | 90.0 | 63.4 | 3-4+ | 93-95 | 4 |
| 1c | 0.47 | 91.4 | 57.3 | 4+ | 94-96 | 4 |
| 1d | 0.32 | 84.5 | 62.7 | 4-5 | 98 | 4 |
| 1e | 0.29 | 85.5 | 61.2 | 4-5+ | 98-99 | 4 |
| 1f | 0.35 | 86.9 | 58.2 | 4-5 | 98 | 4 |
| 1g | 0.3 | 82.3 | 61.8 | 4-5 | 98 | 3-4 |
| 1h | 0.44 | 72.6 | 58.5 | 3-4 | 92-94 | 2-3 |
| 1aa | 0.45 | 87.0 | 58.9 | 4 | 94-96 | 4 |
| 1ab | 0.44 | 88.2 | 63.9 | 4-5 | 97 | 4 |
| 1ac | 0.3 | 90.4 | 65.0 | 4 | 93-95 | 4 |
| 1ad | 0.38 | 91.8 | 62.0 | 4+ | 94-95 | 4+ |
| 1ae | 0.45 | 92.7 | 58.0 | 4 | 93-95 | 4 |
| 1af | 0.42 | 83.8 | 59.3 | 3-4+ | 92-94 | 4 |
| 1ag | 0.36 | 84.4 | 62.7 | 4 | 95-97 | 4 |
| 1ah | 0.40 | 87.5 | 59.3 | 4-5 | 97-99 | 4 |
| 1ai | 0.36 | 93.6 | 57.9 | 4+ | 91-93 | 3-4 |
| 1aj | 0.26 | 91.4 | 63.8 | 4+ | 94-96 | 3-4 |
| 1ak | 0.4 | 91.2 | 51.5 | 4+ | 93-95 | 3+ |
| 1ap | 20.0 | 83.7 | 51.2 | 2 | ~55 | 3 |
| 1ar | 18.0 | 86.4 | 60.5 | 4-5 | 98 | 2-3+ |
| 1at | 0.42 | 86.3 | 58.1 | 3-4 | 90-92 | 3+ |
| 1bi | 0.48 | 85.6 | 58.5 | 4-5 | 95-96 | 3-4 |
| 1bj | 0.31 | 83.2 | 61.0 | 3-4 | 84-86 | 3-4 |

### Discussion

It can be seen that the 4,4′-diazobenzanilide derivatives 1A are dyes of yellow or orange shade (°Hue values ranging from 72.6 to 92.7). *C values of up to 65 confirm the good brilliance associated with such structures. A backwater of 11s highly coloured, whereas a backwater of 5 is colourless. As can be seen the dyes of the present invention yield almost colourless backwater and thus show a high substantivity. The maximum degree of exhaustion is 100%. A degree of exhaustion of above 95% can be regarded as excellent, and a degree of exhaustion of above 90% can be regarded as very good. A lightfastness of 1 is very bad, whereas a lightfastness of 8 is the best possible lightfastness. On paper lightfastnesses usually never exceed 6.5, thus the dyes of the present invention exhibit good to very good lightfastnesses.

1. A 4,4′-diazobenzanilide derivative of the formula 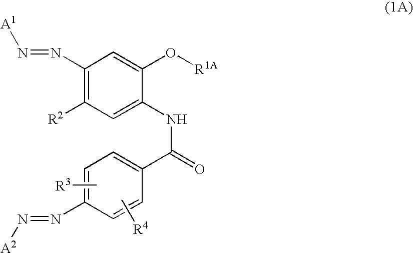
in which
A¹ represents phenyl or 1- or 2-naphthyl, whereby phenyl can be unsubstituted or mono- or disubstituted with sulfo, C₁₋₄-alkyl, C₁₋₄-alkoxy, C₂₋₄-hydroxyalkoxy, halogen, hydroxy, amino, acetamido, ureido or carboxy, and whereby 1- or 2-naphthyl can be unsubstituted or substituted with one or more sulfo groups, and
A² represents a residue selected from the group consisting of 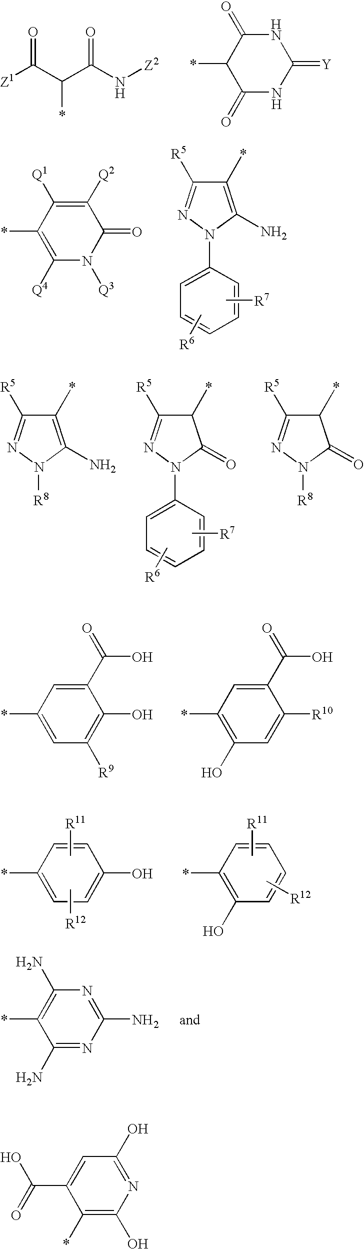
in which
Z¹ represents C₁₋₄-alkyl or phenyl, whereby phenyl may be unsubstituted or mono-substituted with C₁₋₄-alkyl, C₁₋₄-alkoxy or halogen, and
Z² represents phenyl or 1- or 2-naphthyl, whereby phenyl may be unsubstituted or mono-, di- or trisubstituted with sulfo, C₁₋₄-alkyl, C₁₋₄-alkoxy, C₂₋₄-hydroxyalkoxy, halogen, hydroxy, amino, acetamido, ureido or carboxy and whereby 1- or 2-naphthyl may be unsubstituted or mono- or disubstituted with sulfo or carboxy,
Y represents O, N—CN or N—CONH₂,
Q¹ represents hydrogen, hydroxy, C₁₋₂-alkyl, hydroxyethyl, C₁₋₂-alkoxy, carboxy, carbamoyl, C₁₋₂-alkoxycarbonyl, and
Q² represents hydrogen, cyano, halogen, sulfo, C₁₋₂-alkyl, or carbamoyl whereby C₁₋₂-alkyl may be unsubstituted or substituted with hydroxy, phenyl or sulfo, and
Q³ represents hydrogen, phenyl, C₁₋₂-alkylphenyl, C₁₋₄-alkyl, whereby C₁₋₄-alkyl may be unsubstituted or substituted with hydroxy, cyano, C₁₋₂-alkoxy or sulfo, and
Q⁴ represents hydrogen or hydroxy,
R⁵ represents hydrogen, C₁₋₄-alkyl, C₂₋₄-alkenyl, carboxy, NHCOC₁₋₄-alkyl, and
R⁶ and R⁷ each independently from each other represent hydrogen, halogen, sulfo, C₁₋₄-alkyl or carboxy, and
R⁸ represents hydrogen or C₁₋₄-alkyl,
R⁹ represents hydrogen, C₁₋₄-alkyl, and
R¹⁰ represents hydrogen or hydroxy,
R¹¹ and R¹² each independently from each other represent hydrogen, C₁₋₄-alkyl, C₁₋₄-alkoxy, hydroxy, halogen, amino, acetamido, sulfo, carboxy, C₁₋₄-alkoxycarbonyl or C₁₋₄-alkylaminocarbonyl, and
R² represents hydrogen, C₁₋₄-alkyl, C₁₋₄-alkoxy, halogen, hydroxy, carboxy, acetamido, ureido or sulfo, whereby C₁₋₄-alkyl and C₁₋₄-alkoxy may be unsubstituted or substituted with halogen, hydroxy, carboxy, acetamido, ureido or sulfo, and
R³ and R⁴ each independently from each other represent hydrogen, C₁₋₄-alkyl, C₁₋₄-alkoxy, halogen, hydroxy, carboxy, amino, C₁₋₄-alkylamino, acetamido or ureido, whereby C₁₋₄-alkyl and
C₁₋₄-alkoxy may be unsubstituted or substituted with halogen, hydroxy, carboxy, amino, C₁₋₄-alkylamino, acetamido or ureido, and
R^{1A} represents a residue selected from the group consisting of 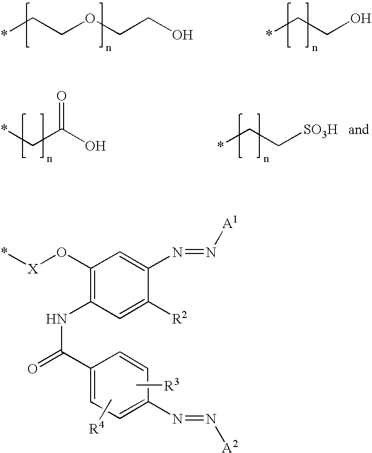
in which
n≧1,
A¹, A², R², R³ and R⁴ have the meaning as indicated above, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S.

2. A 4-amino-4′-azobenzanilide derivative of the formula 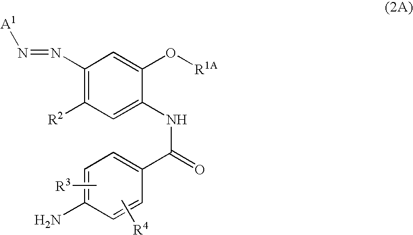
in which A¹, R², R³ and R⁴ have the meaning as indicated in claim 1, and
R^{1A} represents a residue selected from the group consisting of 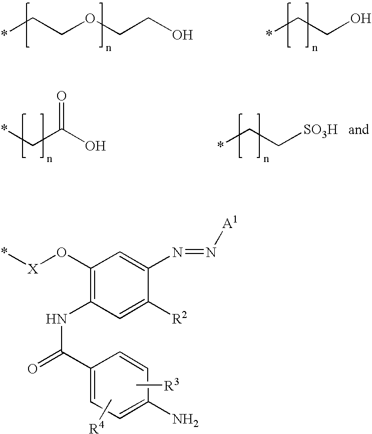
in which
n≧1,
A¹, R², R³ and R⁴ have the meaning as indicated above, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S.

3. A process for the preparation of a 4-amino-4′-azobenzanilide derivative of the formula 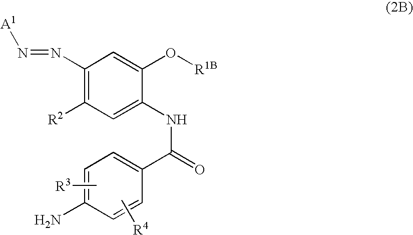
in which A¹, R², R³ and R⁴ have the meaning as indicated in claim 1, and
R^{1B} represents a residue selected from the group consisting of 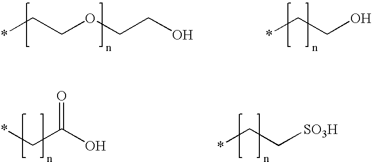
in which
n≧1,
comprising the steps of
i) reacting a 2-nitrophenol derivative of the formula 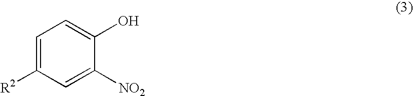
with a compound of the formula
R^{1B}-LG (4B)
in which LG represents a leaving group, to yield a nitrobenzol derivative of the formula 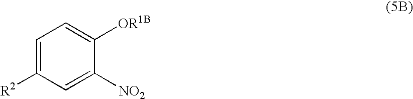
ii) reducing the nitrobenzol derivative of formula 5B obtained in step i) to yield an aniline derivative of the formula 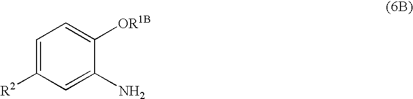
i) diazotizing an amine of the formula
A¹-NH₂ (7)
to yield a diazonium ion of the formula
A¹—N⁺≡N (8)
ii) coupling the diazonium ion of the formula 8 obtained in step iii) with the aniline derivative of formula 6B obtained in step ii) to yield a coupling product of the formula 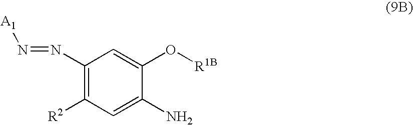
v) reacting the coupling product of formula 9B obtained in step iv) with a nitrobenzoylchloride derivative of the formula 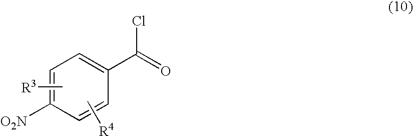
to yield a nitro compound of the formula 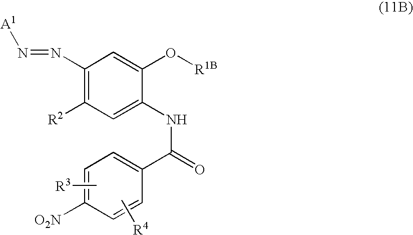
vi) reducing the nitro compound of the formula 11B obtained in step v) to yield the 4-amino-4′-azobenzanilide derivative of formula 2B.

4. A process for the preparation of a 4-amino-4′-azobenzanilide derivative of the formula 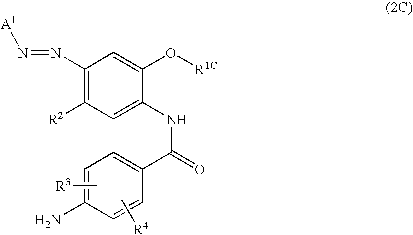
in which A¹, R², R³ and R⁴ have the meaning as indicated in claim 1, and R^{1C} represents 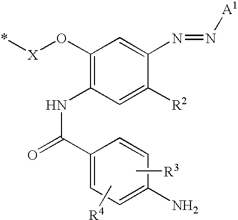
in which
A¹, R², R³ and R⁴ have the meaning as indicated in claim 1, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S,
comprising the steps of
i) reacting a 2-nitrophenol derivative of the formula 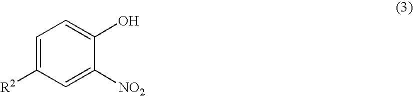
with a compound of the formula 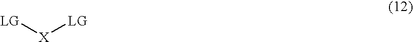
in which LG represents a leaving group, to yield a nitrobenzol derivative of the formula 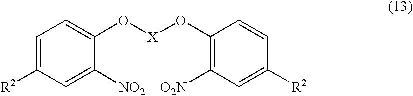
ii) reducing the nitrobenzol derivative of formula 13 obtained in step i) to yield an aniline derivative of the formula 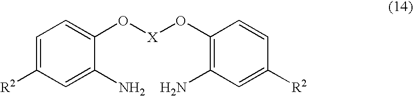
iii) diazotizing an amine of the formula
A¹—NH₂ (7)
to yield a diazonium ion of the formula
A¹—N⁺≡N (8)
iv) coupling the diazonium ion of the formula 8 obtained in step iii) with the aniline derivative of formula 14 obtained in step ii) to yield a coupling product of the formula 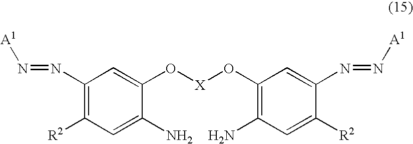
v) reacting the coupling product 15 obtained in step iv) with a nitrobenzoylchloride derivative of the formula 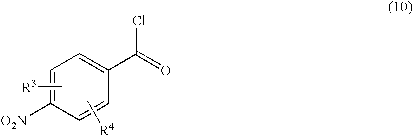
to yield a nitro compound of the formula 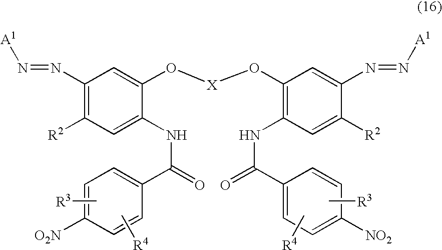
vi) reducing the nitro compound 16 obtained in step v) to yield the 4-amino-4′-azobenzanilide derivative 2C.

5. A process for the preparation of a 4,4′-diazobenzanilide derivative of the formula 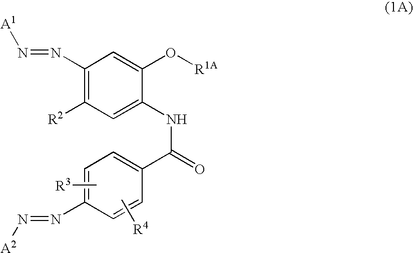
in which A¹, A², R^{1A}, R², R³ and R⁴ have the meaning as indicated in claim 1
comprises the steps of
i) diazotizing a 4-amino-4′-azobenzanilide derivative of the formula 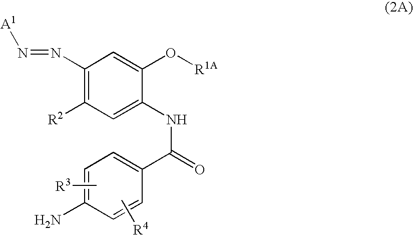
to yield a diazonium ion of the formula 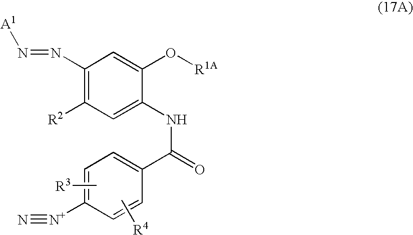
in which A¹, R², R³ and R⁴ have the meaning as indicated in claim 1 and R^{1A} represents a residue selected from the group consisting of 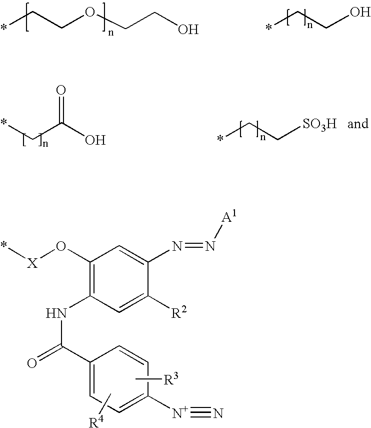
in which
n≧1,
A¹, A², R², R³ and R⁴ have the meaning as indicated in claim 1, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S,
ii) coupling the diazonium ion 17A obtained in step i) with a compound of the formula
A²-H (18)
in which A² has the meaning as indicated in claim 1 to yield the 4,4′-diazobenzanilide derivative 1A.

6. The process for the preparation of the 4,4′-diazobenzanilide derivative of formula (1A) 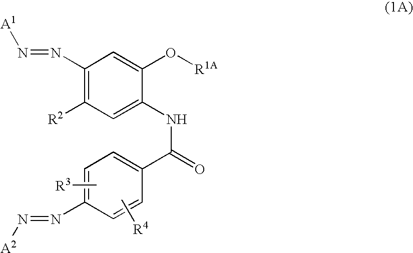
in which
A¹ represents phenyl or 1- or 2-naphthyl, whereby phenyl can be unsubstituted or mono- or disubstituted with sulfo, C₁₋₄-alkyl, C₁₋₄-alkoxy, C₂₋₄-hydroxyalkoxy, halogen, hydroxy, amino, acetamido, ureido or carboxy, and whereby 1- or 2-naphthyl can be unsubstituted or substituted with one or more sulfo groups, and
A² represents a residue selected from the group consisting of 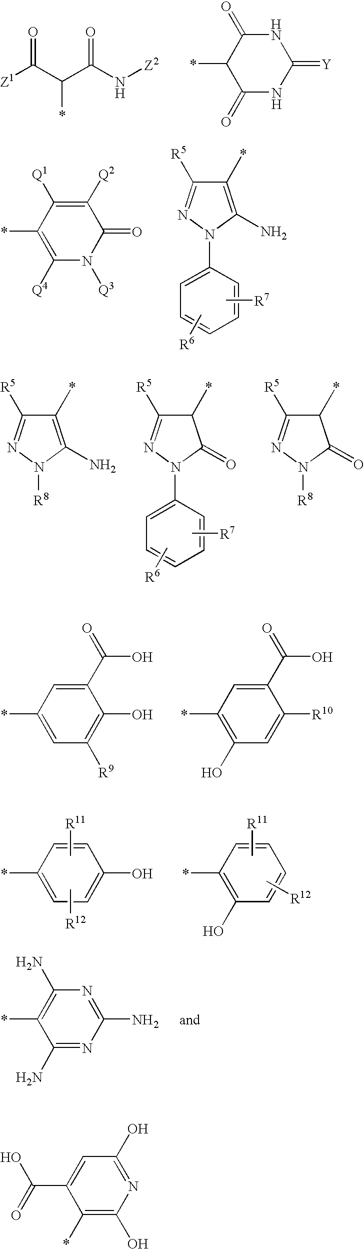
in which
Z¹ represents C₁₋₄-alkyl or phenyl, whereby Phenyl may be unsubstituted or mono-substituted with C₁₋₄-alkyl, C₁₋₄-alkoxy or halogen, and
Z² represents phenyl or 1- or 2-naphthyl, whereby phenyl may be unsubstituted or mono-, di- or trisubstituted with sulfo, C₁₋₄alkyl, C₁₋₄-alkoxy, C₂₋₄-hydroxyalkoxy, halogen, hydroxy, amino, acetamido, ureido or carboxy and whereby 1- or 2-naphthyl may be unsubstituted or mono- or disubstituted with sulfo or carboxy,
Y represents O, N—CN or N—CONH₂,
Q¹ represents hydrogen, hydroxy, C₁₋₂-alkyl, hydroxyethyl, C₁₋₂-alkoxy, carboxy, carbamoyl, C₁₋₂-alkoxycarbonyl, and
Q² represents hydrogen, cyano, halogen, sulfo, C₁₋₂-alkyl, or carbamoyl whereby C₁₋₂-alkyl may be unsubstituted or substituted with hydroxy, phenyl or sulfo, and
Q³ represents hydrogen, phenyl, C₁₋₂-alkylphenyl, C₁₋₄-alkyl, whereby C₁₋₄-alkyl may be unsubstituted or substituted with hydroxy, cyano, C₁₋₂-alkoxy or sulfo, and
Q⁴ represents hydrogen or hydroxy,
R⁵ represents hydrogen, C₁₋₄-alkyl, C₁₋₄-alkenyl, carboxy, NHCOC₁₋₄-alkyl, and
R⁶ and R⁷ each independently from each other represent hydrogen, halogen, sulfo, C₁₋₄-alkyl or carboxy, and
R⁸ represents hydrogen or C₁₋₄-alkyl,
R⁹ represents hydrogen, C₁₋₄-alkyl, and
R¹⁰ represents hydrogen or hydroxy,
R¹¹ and R¹² each independently from each other represent hydrogen, C₁₋₄-alkyl, C₁₋₄-alkoxy, hydroxy, halogen, amino, acetamido, sulfo, carboxy, C₁₋₄alkoxycarbonyl or C₁₋₄-alkylaminocarbonyl, and
R² represents hydrogen, C₁₋₄-alkyl, C₁₋₄-alkoxy, halogen, hydroxy, carboxy, acetamido, ureido or sulfo, whereby C₁₋₄-alkyl and C₁₋₄-alkoxy may be unsubstituted or substituted with halogen, hydroxy, carboxy, acetamido, ureido or sulfo, and
R³ and R⁴ each independently from each other represent hydrogen, C₁₋₄-alkyl, C₁₋₄-alkoxy, halogen, hydroxy, carboxy, amino, C₁₋₄-alkylamino, acetamido or ureido, whereby C₁₋₄-alkyl and C₁₋₄-alkoxy may be unsubstituted or substituted with halogen, hydroxy, carboxy, amino, C₁₋₄-alkylamino, acetamido or ureido, and
R^{1A} represents a residue selected from the group consisting of 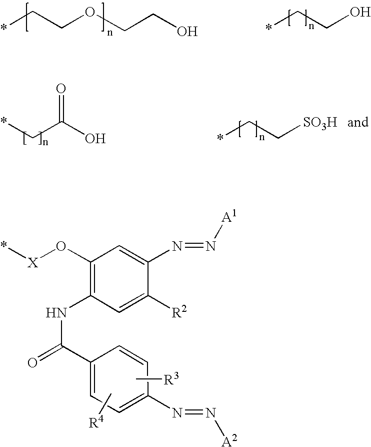
in which
n≧1,
A¹, A², R², R³ and R⁴ have the meaning as indicated above, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S.
wherein the 4-amino-4′-azobenzanilide derivative is prepared according to the process of claim 3.

7. A method of dyeing natural or synthetic materials by applying to the materials the 4,4′-diazobenzanilide derivatives according to claim 1.

8. A method of dyeing paper by applying to the paper the 4,4′-diazobenzanilide derivatives according to claim 1.

9. Paper dyed with a 4,4′-diazobenzanilide derivative according to claim 1.

10. An aqueous formulation comprising a 4,4′-diazobenzanilide derivative according to claim 1.

11. A solid formulation comprising a 4,4′-diazobenzanilide derivative according to claim 1.

12. A process for the preparation of the 4,4′-diazobenzanilide derivative of formula (1A) 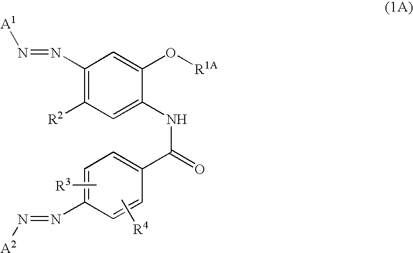
in which
A¹ represents phenyl or 1- or 2-naphthyl, whereby phenyl can be unsubstituted or mono- or disubstituted with sulfo, C₁₋₄-alkyl, C₁₋₄-alkoxy, C₂₋₄-hydroxyalkoxy, halogen, hydroxy, amino, acetamido, ureido or carboxy, and whereby 1- or 2-naphthyl can be unsubstituted or substituted with one or more sulfo groups, and
A² represents a residue selected from the group consisting of 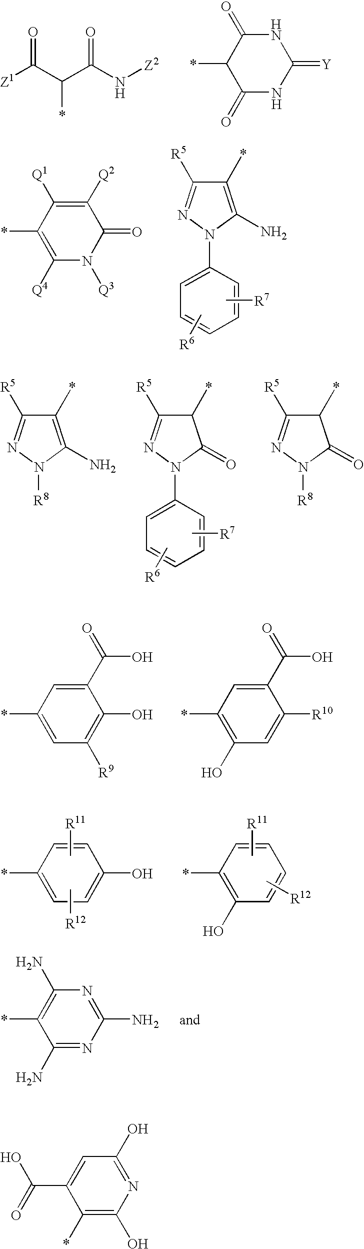
in which
Z¹ represents C₁₋₄-alkyl or phenyl, whereby phenyl may be unsubstituted or mono-substituted with C₁₋₄-alkyl, C₁₋₄-alkoxy or halogen, and
Z² represents phenyl or 1- or 2-naphthyl, whereby phenyl may be unsubstituted or mono-, di- or trisubstituted with sulfo, C₁₋₄-alkyl, C₁₋₄-alkoxy, C₂₋₄-hydroxyalkoxy, halogen, hydroxy, amino, acetamido, ureido or carboxy and whereby 1- or 2-naphthyl may be unsubstituted or mono- or disubstituted with sulfo or carboxy,
Y represents O, N—CN or N—CONH₂,
Q¹ represents hydrogen, hydroxy, C₁₋₂-alkyl, hydroxyethyl, C₁₋₂-alkoxy, carboxy, carbamoyl, C₁₋₂-alkoxycarbonyl, and
Q² represents hydrogen, cyano, halogen, sulfo, C₁₋₂-alkyl, or carbamoyl whereby C₁₋₂-alkyl may be unsubstituted or substituted with hydroxy, phenyl or sulfo, and
Q³ represents hydrogen, phenyl, C₁₋₂-alkylphenyl, C₁₋₄-alkyl, whereby C₁₋₄-alkyl may be unsubstituted or substituted with hydroxy, cyano, C₁₋₂-alkoxy or sulfo, and
Q⁴ represents hydrogen or hydroxy,
R⁵ represents hydrogen, C₁₋₄-alkyl, C₂₋₄-alkenyl, carboxy, NHCOC₁₋₄-alkyl, and
R⁶ and R⁷ each independently from each other represent hydrogen, halogen, sulfo, C₁₋₄-alkyl or carboxy, and
R⁸ represents hydrogen or C₁₋₄-alkyl,
R⁹ represents hydrogen, C₁₋₄-alkyl, and
R¹⁰ represents hydrogen or hydroxy,
R¹¹ and R¹² each independently from each other represent hydrogen, C₁₋₄-alkyl, C₁₋₄-alkoxy, hydroxy, halogen, amino, acetamido, sulfo, carboxy, C₁₋₄-alkoxycarbonyl or C₁₋₄-alkylaminocarbonyl, and
R² represents hydrogen, C₁₋₄-alkyl, C₁₋₄-alkoxy, halogen, hydroxy, carboxy, acetamido, ureido or sulfo, whereby C₁₋₄-alkyl and C₁₋₄-alkoxy may be unsubstituted or substituted with halogen, hydroxy, carboxy, acetamido, ureido or sulfo, and
R³ and R⁴ each independently from each other represent hydrogen, C₁₋₄-alkyl, C₁₋₄-alkoxy, halogen, hydroxy, carboxy, amino, C₁₋₄-alkylamino, acetamido or ureido, whereby C₁₋₄-alkyl and C₁₋₄-alkoxy may be unsubstituted or substituted with halogen, hydroxy, carboxy, amino, C₁₋₄-alkylamino, acetamido or ureido, and
R^{1A} represents a residue selected from the group consisting of 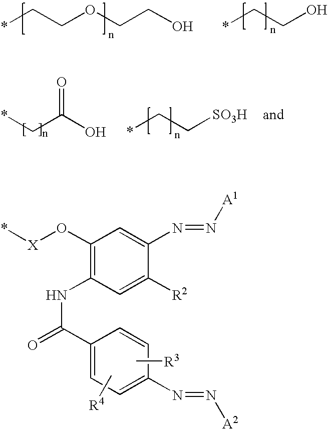
in which
n≧1,
A¹, A², R², R³ and R⁴ have the meaning as indicated above, and
X represents C₂₋₁₄-alkylene, whereby a —CH₂CH₂CH₂— unit of C₂₋₁₄-alkylene may be replaced by a —CH₂-E-CH₂— unit, in which E represents O, NH or S.
wherein the 4-amino-4′-azobenzanilide derivative is prepared according to the process of claim 4.
